SEQUENCE LISTING (12 sequences)

SEQ ID NO: 1 - DNA, 1539 bp, mus musculus
  CDS (1)..(1539)
atggtgcccactggccaggtggcagaaaagcaggcatacgaagagcccaggcaggaccacgaactgaaatcctggcgatgcctggtgttctatctctgcttcttcggcttcatggcccagttgcgtcctggggaaagcttcatcacacccttcctcctggaacgtaaattcaccaaggagcaggtgactaacgagatcattccgatgctgccctactcccacctggctgtgctggtcccggtcttcctgctcaccgactacctgcgatacaagccagtcttggtcctgcagtgcctgagcttcgtgtgcgtgtggctgttgctgctgctgggcacatctgtcgtgcacatgcaactcatggaagtcttctacagcgtcaccatggcggcccgcatcgcctactcctcctacatattctccctggtgcacccctcccgctaccagcgcatggccagctactcacgggcggcagtactgctgggagtcttcatcagctctgtgttgggccaggcgctggtcaccgtaggacatataagcacctatacactcaactgtgtctccctgggcttcatcctcttcagcctggtcctctccctctttctaaagcgccctaagcggagcctctttttcaaccgcagtacgcttgcgcgtggagccttgccctgtgagctggatcagatgcacccggggcctgaccggccagaaaccaggaaactagatcgcatgctgggcacttgcagggactccttcctggtgcgcatgctaagtgaactggtggaaaatgctcggcaaccacagctgcgtctctggtgcctctggtgggtcttcaactcttcaggctactacctgatcacctactatgtgcatgtcctgtggagaagcactgacagcagcctcagctacaatggcgccgtagatgccgcctccacactgctgagcgccatcacgtccttctccgccggcttcctgagcatccgctggactctgtggtccaagctggtcatcgcaggtgtgatcgccatccaggccagcctggttttctgtatgttccagatccgggacatctgggtgtgctacgtgacctttgtgcttttccgtggggcctaccagttccttgtgcccattgccacttttcagattgcgtcttccctgtctaaagagctctgtgcattggtctttgggatcaacactttcctagctactgcgcttaagacctgtatcacacttgtggtctctgacaagcggggtctgggtctccaagtccgtgatcagttccgtatctacttcatatacttcctgatgctgtctatcacctgctttgcctgggcggggctggatggcttaaggtactgccagcgtggtcgccaccagcccctggcccaggcccaggagctgaggagtcccttggagacttctgtgcaagcgataagcctacaggatggagacctaaggggaccgcagccctcagcgccacagctgctctcagaggatggcatggaggacgacaggggagacttaagagtcgaagccaaggcttga

SEQ ID NO: 2 - protein, 512 aa, mus musculus
MVPTGQVAEKQAYEEPRQDHELKSWRCLVFYLCFFGFMAQLRPGESFITPFLLERKFTKEQVTNEIIPMLPYSHLAVLVPVFLLTDYLRYKPVLVLQCLSFVCVWLLLLLGTSVVHMQLMEVFYSVTMAARIAYSSYIFSLVHPSRYQRMASYSRAAVLLGVFISSVLGQALVTVGHISTYTLNCVSLGFILFSLVLSLFLKRPKRSLFFNRSTLARGALPCELDQMHPGPDRPETRKLDRMLGTCRDSFLVRMLSELVENARQPQLRLWCLWWVFNSSGYYLITYYVHVLWRSTDSSLSYNGAVDAASTLLSAITSFSAGFLSIRWTLWSKLVIAGVIAIQASLVFCMFQIRDIWVCYVTFVLFRGAYQFLVPIATFQIASSLSKELCALVFGINTFLATALKTCITLVVSDKRGLGLQVRDQFRIYFIYFLMLSITCFAWAGLDGLRYCQRGRHQPLAQAQELRSPLETSVQAISLQDGDLRGPQPSAPQLLSEDGMEDDRGDLRVEAKA

SEQ ID NO: 3 - DNA, 1776 bp, homo sapiens
  CDS (1)..(1776)
atggtgccctccagcccagcggtggagaagcaggtgcccgtggaacctgggcctgaccccgagctccggtcctggcggcacctcgtgtgctacctttgcttctacggcttcatggcgcagatacggccaggggagagcttcatcaccccctacctcctggggcccgacaagaacttcacgcgggagcaggtcacgaacgagatcacgccggtgctgtcgtactcctacctggccgtgctggtgcccgtgttcctgctcaccgactacctgcgctacacgccggtgctgctgctgcaggggctcagcttcgtgtcggtgtggctgctgctgctgctgggccactcggtggcgcacatgcagctcatggagctcttctacagcgtcaccatggccgcgcgcatcgcctattcctcctacatcttctctctcgtgcggcccgcgcgctaccagcgtgtggccggctactcgcgcgctgcggtgctgctgggcgtgttcaccagctccgtgctgggccagctgctggtcactgtgggccgagtctccttctccacgctcaactacatctcgctggccttcctcaccttcagcgtggtcctcgccctcttcctgaagcgccccaagcgcagcctcttcttcaaccgcgacgaccgggggcggtgcgaaacctcggcttcggagctggagcgcatgaatcctggcccaggcgggaagctgggacacgccctgcgggtggcctgtggggactcagtgctggcgcggatgctgcgggagctgggggacagcctgcggcggccgcagctgcgcctgtggtccctctggtgggtcttcaactcggccggctactacctggtggtctactacgtgcacatcctgtggaacgaggtggaccccaccaccaacagtgcgcgggtctacaacggcgcggcagatgctgcctccacgctgctgggcgccatcacgtccttcgccgcgggcttcgtgaagatccgctgggcgcgctggtccaagctgctcatcgcgggcgtcacggccacgcaggcggggctggtcttccttctggcgcacacgcgccacccgagcagcatctggctgtgctatgcggccttcgtgctgttccgcggctcctaccagttcctcgtgcccatcgccacctttcagattgcatcttctctgtctaaagagctctgtgccctggtcttcggggtcaacacgttctttgccaccatcgtcaagaccatcatcactttcattgtctcggacgtgcggggcctgggcctcccggtccgcaagcagttccagttatactccgtgtacttcctgatcctgtccatcatctacttcttgggggccatgctggatggcctgcggcactgccagcggggccaccacccgcggcagcccccggcccagggcctgaggagtgccgcggaggagaaggcagcacaggcactgagcgtgcaggacaagggcctcggaggcctgcagccagcccagagcccgccgctttccccagaagacagcctgggggctgtggggccagcctccctggagcagagacagagcgacccatacctggcccaggccccggccccgcaggcagctgaattcctgagcccagtgacaaccccttccccctgcactctgtcgtccgcccaagcctcaggccctgaggctgcagatgagacttgtccccagctggctgtccatcctcctggtgtcagcaagctgggtttgcagtgtcttccaagcgacggtgttcagaatgtgaaccagtga

SEQ ID NO: 4 - protein, 591 aa, homo sapiens [sequence omitted]

SEQ ID NO: 5 - DNA, 1539 bp, rattus norvegicus
  CDS (1)..(1539)
atggtgcccactggccaggtcggagaaaagcaagcctgtgaagagcccaggcaggaccgagaacttaagtcctggcgatggctcgtgttctacctctgcttctttggcttcatggcacagttgcgacctggggagagcttcatcacaccctacctcctggaacgtaacttcaccaaggagcaggtgaccaacgagatcattccgatgctgccctactcccacctggccgtgctggtccccatcttcctgctcaccgactacctacgatacaagccagtcctggtcctacagtgcctgagcttcgtgtgcgtgtggctgctgctgctcctggggacctccgtcgtgcacatgcagctcatggaagtcttctacagcatcaccatggcggcgcgcatcgcctactcctcctacatattctccctggtgcagccctcccgctaccagcgcatggccagctactcgcgggccgcagtcctactgggcgtcttcatcagctccgtgctgggccaggtgctggtcaccctaggaggcatctccacctatatgctcaactgtatctccctgggcttcatcctcttcagcctgagcctctccctcttcctaaagcgtcctaagcggagcctctttttcaaccggagcgcacttgtgcaaggagccttgccctgtgagctggatcagatgcacccgggacctggccgtccagagcccaggaaactggagcgcatgctgggcacttgcagggactccttcctggtacgcatgctaagcgaactggtgaagaacgtccggcaaccacagctgcgtctctggtgcctctggtgggtcttcaactcggcaggctactacctgatcacctactatgtccatgtcctgtggaaaatcactgacagcagactcaactacaacggtgccgtagatgccgcctccacacttctgagcgccatcacggccttcaccgcgggcttcgtgaacatccgctgggctctgtggtcaaagctggtcatcgcgagtgtcatagccatccaggctggcctggttttctgtatgttccagatcccggacatctgggtctgctatgtgaccttcgtgcttttccgtggggcctaccagttccttgtgcccattgccacttttcagattgcatcttccctgtctaaagagctctgtgcattggtcttcgggatcaacactttccttgccactgcgcttaagacctcgatcacactcgtggtctctgacaagcggggtctgggcctccaagtccatcaacagttccgcatctacttcatgtacttcctgacgctgtccatcatctgccttgcctgggctgggctggacggcttgaggtactaccggcgtggtcgccaccagccgctggcccaggcccaggctctgagtcccttggaggattctgtgcaggcgataagcctacaggatggagacctaaggagaccgcagccctcggcaccacagctgctcccagaggatggcagcgtagaagacggcagggcagacttgagagtcgaagccaaggcctga

SEQ ID NO: 6 - protein, 512 aa, rattus norvegicus
MVPTGQVGEKQACEEPRQDRELKSWRWLVFYLCFFGFMAQLRPGESFITPYLLERNFTKEQVTNEIIPMLPYSHLAVLVPIFLLTDYLRYKPVLVLQCLSFVCVWLLLLLGTSVVHMQLMEVFYSITMAARIAYSSYIFSLVQPSRYQRMASYSRAAVLLGVFISSVLGQVLVTLGGISTYMLNCISLGFILFSLSLSLFLKRPKRSLFFNRSALVQGALPCELDQMHPGPGRPEPRKLERMLGTCRDSFLVRMLSELVKNVRQPQLRLWCLWWVFNSAGYYLITYYVHVLWKITDSRLNYNGAVDAASTLLSAITAFTAGFVNIRWALWSKLVIASVIAIQAGLVFCMFQIPDIWVCYVTFVLFRGAYQFLVPIATFQIASSLSKELCALVFGINTFLATALKTSITLVVSDKRGLGLQVHQQFRIYFMYFLTLSIICLAWAGLDGLRYYRRGRHQPLAQAQALSPLEDSVQAISLQDGDLRRPQPSAPQLLPEDGSVEDGRADLRVEAKA

SEQ ID NO: 7 - DNA, 1557 bp, cricetulus griseus
  CDS (1)..(1557)
atggtgcccactggccaggtggcagaaaagcaggcatgtgaggagcccaggcaggaccgcgaactcaagtcctggagatgcctagtattctatctctgcttcttcggcttcatggcacagttgcggcctggggagagcttcatcacaccctacctcctgcaacaaaacttcacaatcgagcaggtgaccaatgagatcattccggtgctgccctactcccacctggccgtgctggtgccaatctttctgctcacggactacctgcgatacaagccaatcctgatcctgcagtgcctgagcttcatgtgcgtctggctgctgctgctgctgggcacctctgtggtacacatgcagctcatggaagtcttctacagcgtcaccatggctgcgcgcatcgcctactcctcctacatcttctccctggtgcgcccttcccgctaccagcgcatggccagctactcaagggcagcagtgctcctgggagtcttcaccagctctgtgttgggccaggtgctgtggcccctggaacaaaaatcccagaactccaacatgctcaactacatttccctgggctttatcatcttcagcctgggcctctccctctttctaaagcgtcctaagcacagcctctttttcaaccgcagtgcacttgtgcacaaagccttgccctgcgagctggaccagatgcacccgggccctggccgaccagagcctgggaaactagagcgagtgctgggcagttgcaggaactccttcctggtgtgcatgctaagtgaactggtgggaaatttacgacaaccacacgtgcgcctctggtgcctttggtgggtcttcaactctgcaggctactacctgattgtctactatgtgcacgtcctgtggagtattgacaaaaacctcaactacaatggcgccgtagatgctgcctccacactgctgagcgccatcacatccttctctgcgggcttcgtgaagatccgctgggctctgtggtcaaagctggtcatcgcaagtgttatagccatccaggccggactggttttctgtatgtacatggtccactacgtgacctgggtccacaaaatctgggtactctacatgacctacgtgcttttccgcggggcctaccagttcctcgtgcccattgccacttttcagattgcatcttccctatctaaagagctctgtgccttggtcttcgggatcaatacattccttgccactgcgctcaagactgccatcacgcttgtggtctccgacaaacggggtctgggcctcaaagttgaaaaacagttttgcatctactccgtgtacttcatggtgctttctgtcatctgctttgtcggggccgtgctggatggcgtgaggtactgccggcgtggtcgccaccagcccctgcccctgccccaggagctgagtcccttggagaattctgtgcaggtgccaagtatgcaggatagaggcctagggggactgcagccctcagcaccacagctgctcccagaggatggcgtggaggacagcgaggcaagcttgagagctgaagccaaggcctga

SEQ ID NO: 8 - protein, 518 aa, cricetulus griseus
MVPTGQVAEKQACEEPRQDRELKSWRCLVFYLCFFGFMAQLRPGESFITPYLLQQNFTIEQVTNEIIPVLPYSHLAVLVPIFLLTDYLRYKPILILQCLSFMCVWLLLLLGTSVVHMQLMEVFYSVTMAARIAYSSYIFSLVRPSRYQRMASYSRAAVLLGVFTSSVLGQVLWPLEQKSQNSNMLNYISLGFIIFSLGLSLFLKRPKHSLFFNRSALVHKALPCELDQMHPGPGRPEPGKLERVLGSCRNSFLVCMLSELVGNLRQPHVRLWCLWWVFNSAGYYLIVYYVHVLWSIDKNLNYNGAVDAASTLLSAITSFSAGFVKIRWALWSKLVIASVIAIQAGLVFCMYMVHYVTWVHKIWVLYMTYVLFRGAYQFLVPIATFQIASSLSKELCALVFGINTFLATALKTAITLVVSDKRGLGLKVEKQFCIYSVYFMVLSVICFVGAVLDGVRYCRRGRHQPLPLPQELSPLENSVQVPSMQDRGLGGLQPSAPQLLPEDGVEDSEASLRAEAKA

SEQ ID NO: 9 - DNA, 24 bp, Artificial sequence
  misc_feature (1)..(24): primer
tgccaggtgaccagcatccattgt

SEQ ID NO: 10 - DNA, 24 bp, Artificial sequence
  misc_feature (1)..(24): primer
ggggactttccacaccctaactga

SEQ ID NO: 11 - DNA, 24 bp, Artificial sequence
  misc_feature (1)..(24): primer
tatagactgcaccagctgtgcacg

SEQ ID NO: 12 - DNA, 24 bp, Artificial Sequence
  misc_feature (1)..(24): primer
aaattgaccatgccggccatacgg

(19) United States
(12) Patent Application Publication
(10) Pub. No.: US 2002/0092034 A1
(43) Pub. Date: Jul. 11, 2002

(54) Non-human model of gestational and adult folate deficiency and uses thereof

(76) Inventors: David Patterson, Denver, CO (US); Miles B. Brennan, Denver, CO (US); Ute Hochgeschwender, Oklahoma City, OK (US); Wayne F. Flintoff, London (CA); Heather Sadlish, London (CA); T. Michael Underhill, London (CA); Frederick M.R. Williams, London (CA)

Correspondence Address: SHERIDAN ROSS PC, 1560 BROADWAY, SUITE 1200, DENVER, CO, 80202

(21) Appl. No.: 09/962,290

(22) Filed: Sep. 24, 2001

Related U.S. Application Data
(60) Provisional application No. 60/234,853, filed on Sep. 22, 2000.

(51) Int. Cl. A01K 67/027

(52) U.S. Cl. 800/18; 800/9

(57) ABSTRACT

Disclosed is a genetically modified non-human animal that is a model for defective folate transport. Specifically the non-human animal comprises a genetic modification that results in decreased or absent expression and/or biological activity of the endogenous reduced folate carrier. The animal can be further modified by introduction into the genome of a nucleic acid molecule encoding the human reduced folate carrier. Such an animal can be used in methods to identify compounds useful for treating a variety of conditions associated with defective folate transport, or for the identification of anti-folate compounds useful for treating cancer. The animal can also be used to study the biochemical and molecular events associated with folate transport. Compounds identified using such a model are valuable for therapeutic treatments.

### CROSS-REFERENCE TO RELATED APPLICATIONS

This application claims the benefit of priority under 35 U.S.C. § 119(e) from U.S. Provisional Application Serial No. 60/234,853, filed Sep. 22, 2000, and entitled “Non-Human Model of Gestational and Adult Folate Deficiency”. The entire disclosure of U.S. Provisional Application Serial No. 60/234,853 is incorporated herein by reference.

### FIELD OF THE INVENTION

The present invention relates to a non-human animal model for folate deficiency and to uses of such an animal for studying and developing methods for modifying the effects of folate deficiency and for studying and developing methods for modifying anti-neoplastic agents effecting folate metabolism. In particular, the present invention relates to a reduced folate carrier (RFC) homozygous mutant mouse and uses thereof, and to a transgenic mouse expressing the human rfc gene. In addition, the present invention relates to methods of modifying the effects of folate deficiency in a patient, thereby treating conditions related thereto.

### BACKGROUND OF THE INVENTION

Folate is an essential nutrient. Folate deficiency during gestation is a major cause of birth defects throughout the world and in the United States, contributing to morbidity and mortality. Neural tube closure defects, in particular anencephaly and spina bifida, are common human birth defects (˜1 in 1000). Evidence to date suggests that daily supplementation with 0.4 mg per day prevents >50% of neural tube closure defects when taken prior to conception and during the first trimester. Because mammals are unable to synthesize folates, transport systems have evolved for the efficient intracellular accumulation of folates. Therefore, if there is a differential requirement for folates during development, this might be evident in the expression pattern of genes which encode proteins involved in the transport of folates across the plasma membrane. Disruption of these genes may result in defects in neural tube development. This has been recently demonstrated for one class of folate transporter, the folate binding protein. The major transporter for cellular folates is the reduced folate carrier (also referred to herein as RFC, REFC or SLC19A1), a high affinity, low capacity system.

Antifolate agents are important drugs to treat many cancers. Unfortunately, resistance to these agents by mutation of the human reduced folate carrier gene (RFC) reduces the effectiveness of these drugs. The testing of anti-folate drugs in mouse is confounded by the differences of the pharmacology of mouse and human reduced folate carriers. In order to engineer a mouse mutant suitable for testing human anti-folate drugs, it is necessary both to remove the endogenous mouse reduced folate carrier gene and to introduce the human reduced folate carrier gene into the mouse germline. The introduction of a null allele at the mouse reduced folate carrier gene fulfills the first of the two criteria, while the introduction of a human rfc gene into the mouse germline fulfills the second.

Aside from supplementation with folic acid and, possibly methionine and inositol, there is no preventative therapy for spina bifida and other neural tube closure defects. A number of mouse mutants, both spontaneous and engineered, with neural tube closure defects have been characterized. Surprisingly, at least three of these mutant (Cartl, Crooked and Splotched) respond to dietary supplementation with folic acid by significant decreases in the occurrence of neural tube closure defects.

While the mechanistic significance of folate supplementation is still uncertain, the importance of folate for neural tube closure is evidenced by its ability to ameliorate these defects. There is an undoubted need for mouse mutants whose primary defect is in folate metabolism in order to elucidate the role of folate in neural tube closure.

There is also a need for mouse mutants which express the human RFC, and not the mouse RFC, for the study of anti-folate drugs.

The development of transgenic and “knock-out” animal technology has provided significant advances for obtaining more complete information about complex systems in vivo. By manipulating the expression of gene(s) in vivo, it is possible to gain insight into the roles of such genes in a particular system or to study aspects of the system in a genetically controlled environment. The biochemical activities associated with developmental defect in a small mammal such as the mouse, will allow analysis of the disorder, and conditions related thereto, at molecular and cellular levels that are often impossible to analyze in humans.

### SUMMARY OF THE INVENTION

One embodiment of the present invention relates to a genetically modified non-human animal comprising a genetic modification in at least one allele of the endogenous reduced folate carrier (rfc) gene in the animal, wherein the genetic modification results in a reduction in endogenous reduced folate carrier (RFC) expression or biological activity in the animal. In one embodiment, the genetic modification is in both alleles of the endogenous rfc gene in the animal.

In one aspect, the genetic modification is a modification of nucleotides in a gene encoding reduced folate carrier (RFC) in the animal selected from the group consisting of a deletion of nucleotides from the gene, an insertion of nucleotides in the gene, a substitution of nucleotides for nucleotides in the gene, and an inversion of nucleotides in the gene. In one aspect, the genetic modification comprises a modification within exons 1-4 of the rfc gene in the animal. In another aspect, the genetic modification comprises a modification with a regulatory region of the rfc gene in the animal. In yet another aspect, the genetic modification is a deletion of a nucleic acid sequence within at least one allele of the rfc gene in the animal. In another aspect, the genetic modification is a deletion of both alleles of the rfc gene in the animal, wherein the genetic modification results in an absence of endogenous reduced folate carrier (RFC) biological activity in the animal. In another aspect, the genetic modification is a deletion of at least a portion of a nucleic acid sequence comprising exons 1-4 of the gene encoding RFC.

In one aspect, the non-human animal is a mouse. In this aspect, the genetic modification can be a deletion from the genome of the mouse of a nucleic acid sequence comprising SEQ ID NO: 1.

Yet another embodiment of the present invention relates to a genetically modified non-human animal having human reduced folate carrier (RFC) biological activity, wherein the animal comprises a first genetic modification in two alleles of an endogenous reduced folate carrier (rfc) gene, wherein the first genetic modification results in an absence of endogenous reduced folate carrier expression or biological activity. The animal further comprises a second genetic modification that results in expression by cells in the animal of a nucleic acid molecule comprising a nucleic acid sequence encoding a biologically active human reduced folate carrier (RFC). The first genetic modification can include any of the genetic modifications described above in the first embodiment of the invention.

In one aspect, the nucleic acid sequence encoding human reduced folate carrier comprises SEQ ID NO:3. In another aspect, the nucleic acid sequence encoding human reduced folate carrier is operatively linked to a human transcriptional control sequence. In another aspect, the nucleic acid sequence encoding human reduced folate carrier is operatively linked to a transcriptional control sequence for the endogenous rfc gene. The cells of the non-human animal can express the human reduced folate carrier at a level that is less than, equivalent to, or greater than the level of expression expected for expression of endogenous RFC in a wild-type animal, as desired.

In one aspect of this embodiment, the non-human animal is a mouse. In this aspect, the genetic modification in the endogenous reduced folate carrier (rfc) gene can be a deletion from the genome of the mouse of a nucleic acid sequence comprising SEQ ID NO: 1, and the nucleic acid sequence encoding human reduced folate carrier can comprise SEQ ID NO:3.

Yet another embodiment of the present invention relates to a method to evaluate anti-folate compounds for the treatment of cancer. The method includes the steps of: (a) contacting an anti-folate compound to be evaluated with cells of a genetically modified non-human animal having human reduced folate carrier (RFC) biological activity, wherein the genome of the non-human animal comprises a first genetic modification in two alleles of its endogenous reduced folate carrier (rfc) gene, wherein the first genetic modification results in an absence of endogenous reduced folate carrier activity, and wherein the genome of the animal further comprises a second genetic modification that results in expression by the cells of the animal of a nucleic acid molecule comprising a nucleic acid sequence encoding a biologically active human reduced folate carrier (RFC); and, (b) selecting compounds from (a) that bind to the human RFC expressed by the cells.

In one aspect of the method, the step of contacting comprises administering the compound to the non-human animal in vivo. In this aspect, the step of selecting can include harvesting a cell source selected from the group consisting of a cell sample, a tissue or a body fluid from the non-human animal and measuring cellular uptake of the compound by the cells. The step of selecting can alternatively, or additionally, include harvesting a cell source selected from the group consisting of a cell sample, a tissue or a body fluid from the non-human animal and measuring binding of the compound to the human RFC expressed by the cells. In one aspect, the non-human animal has been induced to grow tumor cells, and the method further comprises a step of selecting compounds from (a) that, when administered to the animal, result in a reduction in growth of the tumor cells.

In another aspect of the method, the step of contacting comprises harvesting a cell source selected from the group consisting of a cell sample, a tissue or a body fluid from the non-human animal, and contacting cells in the cell source with the compound in vitro. In one aspect, the step of selecting comprises selecting compounds from (a) that bind to the human RFC with a greater affinity than a control compound that binds to human RFC. In another aspect, the cells of the non-human animal express the human reduced folate carrier at a level that is less than the level of expression expected for expression of endogenous RFC in a wild-type animal. In another aspect, the human RFC is a mutant human RFC that is known to have reduced binding affinity for at least one known anti-folate compound.

Another embodiment of the invention relates to a method to treat cancer, comprising administering to a patient that has cancer an anti-folate identified by the method described above.

Yet another embodiment of the present invention relates to a method to evaluate anti-folate compounds for the treatment of cancer. The method includes the steps of: (a) contacting an anti-folate compound to be evaluated with cells of a genetically modified non-human animal having human reduced folate carrier (RFC) biological activity, wherein the genome of the non-human animal comprises a first genetic modification in two alleles of its endogenous reduced folate carrier (rfc) gene, wherein the first genetic modification results in an absence of endogenous reduced folate carrier activity, and wherein the genome of the animal further comprises a second genetic modification that results in expression by the cells of the animal of a nucleic acid molecule comprising a nucleic acid sequence encoding a biologically active human reduced folate carrier (RFC); and (b) selecting compounds from (a) that do not bind to the human RFC expressed by the cells, but which are internalized by the cells. Another embodiment is a method to treat cancer, comprising administering to a patient that has cancer an anti-folate identified by this method.

Another embodiment of the present invention relates to a method for studying the molecular and biochemical events associated with folate transport. This method includes the steps of: (a) harvesting cells, tissues or body fluids from a genetically modified non-human animal comprising a genetic modification in at least one allele of the endogenous reduced folate carrier (rfc) gene in the animal, wherein the genetic modification results in a reduction in endogenous reduced folate carrier (RFC) activity in the animal; and (b) evaluating the cells, tissues or body fluids from the genetically modified non-human animal for molecular or biochemical activity associated with reduced folate transport in the non-human animal.

Yet another embodiment of the present invention relates to a method to identify compounds useful for treating a condition associated with defective folate transport in an animal. This method includes the steps of: (a) contacting a compound to be evaluated with cells of a genetically modified non-human animal that has a genome comprising a genetic modification in at least one allele of the endogenous reduced folate carrier (rfc) gene in the animal, wherein the genetic modification results in a reduction in endogenous reduced folate carrier (RFC) activity in the animal; and (b) selecting compounds from (a) that bind to the RFC expressed by the cells.

In one aspect, the step of selecting comprises selecting compounds that bind to the RFC with a higher affinity than the affinity of folate for the RFC.

In another aspect, the genome of the animal comprises a first genetic modification in two alleles of its endogenous reduced folate carrier (rfc) gene, wherein the first genetic modification results in an absence of endogenous reduced folate carrier activity, and a second genetic modification that results in expression by cells in the animal of a nucleic acid molecule comprising a nucleic acid sequence encoding a biologically active human reduced folate carrier (RFC). In this aspect, the step of selecting can include selecting compounds that bind to the human RFC with a higher affinity than the affinity of folate for the human RFC. In one aspect, the human RFC is a mutant human RFC that is known to be associated with the presence of the condition. In this aspect, the compound to be evaluated is a folate analog and the step of selecting comprises selecting folate analogs that bind to the mutant human RFC with an affinity that is greater than the affinity of folate for the mutant human RFC.

The condition can be any condition associated with defective folate transport, including, but not limited to: a neural tube defect, Parkinson's disease, Alzheimer's disease, a cardiovascular disease, stroke, or cleft palate. One embodiment of the invention includes a method to treat a condition associated with defective folate transport, comprising administering to a patient that has the condition a compound identified by this method.

### BRIEF DESCRIPTION OF THE DRAWINGS OF THE INVENTION

FIG. 1A is a schematic diagram of the murine RFC-1 locus, the targeting vector, and the rearranged locus after homologous targeting.

FIG. 1B is a digitized image of an ethidium bromide stain of a gel running PCR reactions with embryonic stem cell DNA (ES) as templates and primers RFC-1 and MB-4.

FIG. 2 is a digitized image of a Southern blot showing SacI-digested DNA from heterozygous and wildtype pups.

FIG. 3 is a digitized image of an agarose gel electrophotogram showing PCR products from tail DNAs of transgenic mice.

FIG. 4 is a digitized image of a gel showing RT PCR products from RNA of an RFC transgenic mouse amplified with human specific rfc primers.

### DETAILED DESCRIPTION OF THE INVENTION

The present invention generally relates to a non-human animal model for studying and developing protocols for modifying folate metabolism. In particular, the present invention relates to a genetically modified non-human animal which has a genetic modification that results in a reduction, and preferably absence, of reduced folate carrier (RFC) expression and/or biological activity in the animal, and the use of such an animal for studying and developing protocols for studying folate metabolism.

More particularly, the present inventors disclose herein the development and characterization of a RFC mutant mouse which is a model of folate deficiency, and which can be used to study and develop protocols for the treatment of a variety of conditions related to folate related birth defects, conditions that can be affected by folate and folate transport, and conditions in which anti-folates are useful. The RFC mutant mouse was engineered to carry an autosomal recessive null allele of the rfc gene (i.e., the gene encoding the reduced folate carrier). This mouse lacks the transporter encoded by the rfc locus. The present inventors have discovered that mice lacking the RFC have a developmental defect.

Furthermore, the present inventors have demonstrated that the RFC null mutant mouse is a model for studying the human folate deficiency syndromes. In addition to having a mouse model for the human RFC deficiency, the RFC mutant mouse is a valuable addition to the growing number of murine developmental models, aiding in the dissection of the mechanisms of spinal cord closure.

Finally, the present inventors have expressed the human RFC in the mouse that is deficient in endogenous RFC. This mouse is valuable for the evaluation of anti-folate compounds and folate analogs for use in human therapy, such as for the treatment of cancer.

Accordingly, one embodiment of the present invention relates to a genetically modified non-human animal that has decreased reduced folate carrier (RFC) expression and/or biological activity. Such an animal is useful, for example, for studying folate metabolism, for evaluating mechanisms related to folate transport, for evaluating folate and anti-folate compounds, and/or for the expression of a human RFC.

In one embodiment, the genetically modified non-human animal of the present invention comprises a genetic modification within at least one allele of its rfc gene, wherein the genetic modification results in a reduction in reduced folate carrier (RFC) protein expression and/or biological activity in the animal (e.g., a heterozygous mutant animal). In one aspect, the genetic modification includes, but is not limited to, a deletion, an insertion, a substitution and/or an inversion of nucleotides in the rfc gene which results in a reduction in RFC expression and/or biological activity in the animal. The genetic modification can be a modification including or within exons 1-4 of the rfc gene which results in a reduction in RFC expression and/or biological activity, and/or a modification in a region of the rfc gene other than exons 1-4 which results in a reduction in RFC expression and/or biological activity (e.g., a modification in exon 5, exon 6 and/or a regulatory region of the rfc gene). In a preferred embodiment, the genetic modification is a deletion of a nucleic acid sequence within at least one allele of the rfc gene, wherein the deletion results in a reduction of expression of RFC and/or of biological activity by the animal.

In another embodiment, the animal comprises a genetic modification within two alleles (i.e., both alleles) of the rfc gene, wherein the genetic modification results in an absence of RFC expression and biological activity in the animal (e.g., the resulting animal is a homozygous mutant animal). Preferably, the genetic modification is a deletion of a nucleic acid sequence within both alleles of the rfc gene, wherein the deletion results in an absence of expression (and therefore of biological activity) of RFC by the animal. It is noted that in this embodiment, when there is no endogenous RFC expression or activity, the animal will not be viable unless another source of RFC is provided, such as by introduction of the human RFC in to the animal, as described below. Therefore, in this embodiment, unless it is desired to study the embryonic development of a homozygous mutant mouse in the absence of RFC (e.g., before death of the embryo), this embodiment typically includes the introduction of another source of RFC, such as a mutated RFC (but still functional) or an RFC from another animal species.

According to the present invention, the term rfc locus refers to the site on a chromosome that contains the rfc gene. A gene is a nucleic acid molecule that includes the nucleic acid sequence that encodes a specific protein (the coding region) as well as the untranslated regions, including regulatory regions, that flank the coding region. Reference to an rfc gene refers to the gene that contains the coding region for a reduced folate carrier protein. An rfc gene for a particular species of animal may have other names. For example, the human rfc gene is identified by locus name on chromosome 21 as SLC19A1. The nucleic acid and amino acid sequences for the naturally occurring RFC in a large variety of animals (i.e., human, mouse, rat, hamster, etc.) are known in the art. Such sequences can be found, for example, in a protein or nucleic acid database such as GenBank.

For example, the genomic nucleic acid sequence encoding mouse reduced folate carrier can be obtained under GenBank Accession No. AH006781; the mRNA containing the complete coding region for mouse RFC is GenBank Accession No. L23755 (represented herein by SEQ ID NO:1), which encodes the amino acid sequence for RFC (represented herein by SEQ ID NO:2).

The genomic nucleic acid sequence encoding human reduced folate carrier can be obtained under GenBank Accession No. AL163302; the mRNA containing the complete coding region for human RFC is GenBank Accession No. U17566 (represented herein by SEQ ID NO:3), which encodes the amino acid sequence for RFC (represented herein by SEQ ID NO:4). It is noted that the human rfc locus, which is on chromosome 21, is identified by the name, SLC19A1.

The mRNA containing the complete coding region for rat reduced folate carrier is GenBank Accession No. U38180 (represented herein by SEQ ID NO:5), which encodes the amino acid sequence for RFC (represented herein by SEQ ID NO:6).

The mRNA containing the complete coding region for hamster reduced folate carrier is GenBank Accession No. U03031 (represented herein by SEQ ID NO:7), which encodes the amino acid sequence for RFC (represented herein by SEQ ID NO:8).

Another embodiment of the present invention relates to a genetically modified non-human animal that has decreased, and preferably an absence of, endogenous reduced folate carrier (RFC) expression and/or biological activity, and that additionally expresses a human reduced folate carrier protein. An endogenous reduced folate carrier refers to the reduced folate carrier that is naturally expressed by an animal (e.g., by the wild-type animal). A non-human animal that expresses the human reduced folate carrier is useful, for example, for studying human folate metabolism, for evaluating mechanisms related to human folate transport, and particularly, for evaluating folate and anti-folate compounds that may be useful in human therapies (i.e., by acting on or with the human reduced folate carrier).

In addition to the genetic modifications described above for the non-human animal with decreased RFC expression and/or biological activity, the genetically modified non-human animal comprises a genetic modification whereby the animal expresses a nucleic acid sequence encoding human RFC (SEQ ID: 4), or a biologically active fragment of human RFC. Preferably, the animal expresses a nucleic acid sequence comprising SEQ ID NO:3, or a fragment thereof that encodes a biologically active RFC protein. The genetic modification includes an insertion of at least the coding region of the human rfc gene (or a fragment of the coding region that encodes a biologically active RFC) at any location of the mouse genome compatible with its expression. In a preferred embodiment, the coding region is under transcriptional control (i.e., is operatively linked to a transcription control sequence) of the human genomic sequences (i.e., the human regulatory regions). In another embodiment, the human rfc coding sequences are under transcriptional control by the genome of the non-human animal, and preferably, by the regulatory region of the non-human animal's rfc gene (i.e., the regulatory region for the endogenous rfc gene in the non-human animal).

According to this embodiment of the present invention, to express the human RFC or a biologically active fragment thereof refers to the expression of nucleic acids encoding the human RFC in such a manner that cells of the non-human animal express a human RFC having biological activity. In general, the biological activity or biological action of a protein refers to any function(s) exhibited or performed by the protein that is ascribed to the naturally occurring form of the protein as measured or observed in vivo (i.e., in the natural physiological environment of the protein) or in vitro (i.e., under laboratory conditions). Modifications of a protein, such as in a homologue (including a fragment of a full-length protein) or mimetic (both discussed below), may result in proteins having the same biological activity as the naturally occurring protein, or in proteins having decreased or increased biological activity as compared to the naturally occurring protein. Modifications which result in a decrease in protein expression or a decrease in the activity of the protein, can be referred to as inactivation (complete or partial), down-regulation, or decreased action of a protein. Similarly, modifications which result in an increase in protein expression or an increase in the activity of the protein, can be referred to as amplification, overproduction, activation, enhancement, up-regulation or increased action of a protein.

As used herein, a protein that has “reduced folate carrier biological activity” or that is referred to as a “reduced folate carrier” refers to a protein that has an activity that can include any one, and preferably more than one, of the following characteristics: an ability to bind to folate or to folic acid, and an ability to transport compounds that bind to the carrier into a cell that expresses the carrier. Binding can be measured by any standard binding assay. For example, membranes can be harvested from cells expressing RFC by standard techniques and used in an in vitro binding assay. ¹²⁵I-labeled (other labels can be used also) ligand (e.g., ¹²⁵I-labeled folate) is contacted with the membranes and assayed for specific activity; specific binding is determined by comparison with binding assays performed in the presence of excess unlabeled ligand. Membranes are typically incubated with labeled ligand in the presence or absence of test compound. Compounds that bind to the carrier and compete with labeled ligand for binding to the membranes reduced the signal compared to the vehicle control samples. The ability to transport compounds into a cell can be measured using any assay that measures the uptake of a compound into a cell. For example, radioisotope-labeled ligand (e.g., folate, methotrexate) is contacted with a cell that expresses the RFC and uptake of the labeled ligand by the cell is measured, for example by scintillation counting.

One advantage of this embodiment of the present invention is that the expression level of the human reduced folate carrier by the cells of the non-human animal can be varied, as desired, to provide a model system for testing various compounds. For example, decreased expression of the reduced folate carrier in humans is one explanation for impaired methotrexate transport (an anti-folate) in cancer therapy. Therefore, to have a non-human animal model that mimics the decreased level of RFC expression observed in some patients would be valuable for screening for new anti-folate compounds that are taken up at an effective level even in the presence of decreased RFC expression. Accordingly, it is an aspect of the present invention to provide a genetically modified non-human animal that has decreased, equivalent, or increased expression levels of human RFC as compared to the expression level of the endogenous RFC in the wild-type (not genetically modified) animal. For example, the expression level of the human RFC in the genetically modified non-human animal (e.g., which does not express endogenous RFC—homozygous null animal) can be adjusted to approximate the expression of the endogenous RFC by the reference cells in a wild-type animal of the same species as the genetically modified animal. Alternatively, the expression of the human RFC can be adjusted to be lower than that of the endogenous RFC, or to mimic the expression level of human RFC by a human cell type in a methotrexate resistant human patient.

It may be appreciated by one skilled in the art that use of recombinant DNA technologies can improve, reduce or otherwise control expression of exogenous nucleic acid molecules expressed in a host cell by manipulating, for example: the duration of expression of the gene or sequence (i.e., the sequence encoding the human RFC), the number of copies of the nucleic acid molecules within a host cell, the efficiency with which those nucleic acid molecules are transcribed, the efficiency with which the resultant transcripts are translated, and the efficiency of post-translational modifications. Recombinant techniques useful for increasing the expression of nucleic acid molecules of the present invention include, but are not limited to, operatively linking nucleic acid molecules to high-copy number plasmids or targeting vectors, integration of the nucleic acid molecules into one or more host cell chromosomes, addition of vector stability sequences to plasmids, increasing the duration of expression of the transfected molecule, substitutions or modifications of transcription control signals (e.g., promoters, operators, enhancers) to effect the expression, substitutions or modifications of translational control signals (e.g., ribosome binding sites, Kozack consensus sequences), modification of nucleic acid molecules to correspond to the codon usage of the host cell, and deletion of sequences that destabilize transcripts.

In addition, one can modify the human RFC nucleic acid sequence to test for the effects of different mutations on folate and anti-folate uptake. Additionally, to the extent that certain mutations in the human RFC are identified as contributing to impaired folate uptake or to anti-folate resistance in human therapy (e.g., human cancer therapy), one can express these mutated RFCs in the non-human animal model of the present invention in order to identify folate or anti-folate compounds that have good or improved cellular uptake using the mutated RFC. Such assays would be valuable, for example, for identifying anti-folate compounds that are useful for cancer therapy, and which are effectively taken up by cells, even when the RFC is mutated and has shown resistance to standard methotrexate therapy.

As used herein, a non-human animal suitable for genetic modification according to the present invention is any non-human animal for which the rfc gene can be manipulated, including non-human members of the Vertebrate class, Mammalia, such as non-human primates and rodents. Preferably, such a non-human animal is a rodent, and more preferably, a mouse. Genetically modified mice which have either a reduction or an absence of RFC expression are described in detail herein. In addition, such genetically modified mice which have been further modified to express the human RFC are also described.

According to the present invention, a “genetically modified” animal, such as any of the preferred non-human animals described herein, has a genome which is modified (i.e., mutated or changed) from its normal (i.e., wild-type or naturally occurring) form such that the desired result is achieved (e.g., a reduction in the expression and/or biological activity of RFC). Genetic modification of an animal is typically accomplished using molecular genetic and cellular techniques, including manipulation of embryonic cells and DNA (e.g., DNA comprising the rfc gene), or manipulation of somatic cells (e.g., by nuclear transfer cloning techniques). Such techniques are generally disclosed for mice, for example, in “Manipulating the Mouse Embryo” (Hogan et al., Cold Spring Harbor Laboratory Press, 1994, incorporated herein by reference in its entirety), and in several publications describing nuclear transfer cloning techniques (e.g., Wilmut et al., 1997, Nature 385:810-813; Schneike et al., 1997, Science 278:2130-2133; Fluka et al., 1998, Bioessays 20:847-851; Hosaka et al., 2000, Hum. Cell 13:237-242; and Sato et al., 2000, Human Cell 13(4):197-202).

A genetically modified non-human animal can include a non-human animal in which nucleic acid molecules have been modified (i.e., mutated; e.g., by insertion, deletion, substitution, and/or inversion of nucleotides), in such a manner that the modifications provide the desired effect within the animal (e.g., reduction in endogenous RFC transport, expression and/or other biological activity of the human RFC). As used herein, genetic modifications which result in a reduction in gene expression, in the function of the gene, or in the function of the gene product (i.e., the protein encoded by the gene) can be referred to as inactivation (complete or partial), deletion, interruption, blockage or down-regulation of a gene. For example, a genetic modification in a gene which results in a decrease in the function of the protein encoded by such gene, can be the result of: a partial or complete deletion of the gene or of an exon within the gene (i.e., the gene does not exist, and therefore the protein cannot be produced); a mutation (e.g., a deletion, substitution, insertion and/or inversion) in the gene which results in incomplete or no translation of the protein (e.g., a mutation which causes a frame shift so that the correct protein is not expressed, a mutation in one or more exons of the gene so that the protein or at least a portion of the protein is not expressed, or a mutation in a regulatory region so that the protein is not expressed or has reduced expression); or a mutation in the gene which decreases or abolishes the natural function of the protein (e.g., a protein is expressed which has decreased or no biological activity or action).

According to the present invention, a genetic modification of a non-human animal results in a reduction (i.e., decrease, inhibition, down-regulation) of the biological activity of endogenous RFC and expression of the human rfc gene. Such a genetic modification includes any type of modification to a genome of the animal, particularly including modifications made at the embryonic stage of development of the animal (or in the ancestor of the animal). Such modifications are described above. According to the present invention, reference to reducing the biological activity (or action) of endogenous RFC refers to any genetic modification in the non-human animal which results in decreased functionality of RFC, including: decreased binding of folates, anti-folates, or analogues thereof to the RFC, and decreased transport of such compounds into a cell expressing the RFC. For example, the expression and/or biological activity of endogenous RFC can be decreased by blocking or reducing the production of the protein, “knocking out” the gene or a portion of the gene encoding the protein, reducing transport activity, or inhibiting the activity of the transporter. In a preferred embodiment, the genetically modified non-human animal of the present invention has reduced RFC expression and biological activity due to the deletion of one or both rfc alleles.

According to one aspect the present invention, a genetic modification of a non-human animal includes the introduction of the human rfc gene into the genome of the animal such that the human RFC is expressed by cells of the non-human animal. Such a genetic modification includes any type of modification to a genome of the animal, particularly including modifications made at the embryonic stage of development of the animal (or in the ancestor of the animal), and including modification of somatic cells followed by nuclear transfer cloning techniques. According to the present invention, reference to “expressing” the human rfc gene refers to any genetic modification of the non-human animal which results in the introduction of the nucleic acid sequence for human rfc into the non-human animal such that the human RFC protein is expressed by cells of the non-human animal. For example, the endogenous murine rfc gene (SEQ ID: 1) can be modified by incorporation of elements from the human rfc gene (SEQ ID: 3). In another example, the entirety of the human rfc gene (SEQ ID: 3) is integrated at any site of the non-human animal model that is compatible with its expression.

In one embodiment of the present invention, a non-human animal of the present invention is genetically modified by modification of a nucleic acid sequence within one (i.e., heterozygous) or both (i.e., homozygous) alleles of the rfc gene, wherein such modification can include, but is not limited to, a deletion, an insertion, a substitution and/or an inversion within the one or more nucleotides in the rfc gene. In one embodiment, the genetic modification is in a nucleic acid sequence that includes exons 1-4 of the rfc gene (with reference to the mouse sequence, and to homologous sequences for other animal species), such modification resulting in a decrease in RFC expression and/or biological activity in the animal. In another embodiment, the genetic modification is in a region of the rfc gene other than exons 1-4, whereby the modification results in a decrease in RFC expression and/or biological activity in the animal. Such other regions include exon 5, exon 6, or a regulatory region of the rfc gene (again, with reference to the mouse sequence, and to homologous sequences for other animal species). According to the present invention, a regulatory region of a gene includes any regulator sequences that control the expression of nucleic acid molecules, including promoters, enhancers, transcription termination sequences, sequences that regulate translation, and origins of replication.

In a preferred embodiment of the present invention, a non-human animal of the present invention is genetically modified by deletion of a nucleic acid sequence within one or both alleles of the rfc gene, wherein the deletion results in a reduction or absence, respectively, of expression of RFC in the animal. In one embodiment, such a genetic modification is a deletion of a nucleic acid sequence comprising exons 1-4 of RFC. In another embodiment, the genetic modification is a deletion consisting of exons 1-4 of RFC. In yet another embodiment, the genetic modification is a deletion of a portion of exons 1-4 of RFC sufficient to reduce or prevent expression of RFC by at least one allele and more preferably, by both alleles, of the rfc gene of the animal.

In one embodiment of the present invention, the genetically modified non-human animal is a mouse, also referred to herein as an RFC mutant mouse. A mouse having a genetic modification in one allele of the rfc gene (i.e., the other allele is wild-type, or unmodified) can be referred to herein as an RFC heterozygous mutant mouse; a mouse having a genetic modification in both alleles of the rfc gene can be referred to herein as an RFC homozygous mutant mouse or an RFC null mutant mouse, or some combination of such terms. In this embodiment, the genetic modification is preferably a deletion from the genome of a nucleic acid sequence comprising SEQ ID NO: 1, although any genetic modification of the gene as described above is encompassed by the present invention. SEQ ID NO: 1 includes exons 1-4 of the mouse (i.e., Mus musculus) rfc gene and can be located in the GenBank database as GenBank Accession No. L23755. The genomic sequence for mouse rfc is found under GenBank Accession No. AH006781. Preferably, the genetic modification in the mouse is a deletion from the genome of exons 1-4 of RFC.

According to the present invention, a non-human animal can be genetically modified by any method which results in the desired effect (i.e., reduction in RFC expression and/or biological activity in the animal). Such methods are typically molecular techniques, and include, but are not limited to, any deletion of at least a portion of the rfc gene in the animal, any insertion of a non-RFC sequence into at least a portion of the rfc gene in the animal, or any substitution of at least a portion of the rfc gene in the animal with any non-RFC sequence or mutated RFC sequence, sufficient to reduce RFC expression and/or biological activity in the animal. For example, a rfc gene in the genome of an animal (or an embryonic cell) can be genetically modified by inserting into at least one allele of the rfc gene of the animal or cell an isolated nucleic acid molecule which encodes at least a section of a mutated rfc gene. At least a portion of this isolated section of the rfc gene is mutated (i.e., by deletion of the portion, substitution of the portion with another, non-RFC sequence, or insertion of a non-RFC sequence into the section of RFC), such that when the isolated nucleic acid molecule is inserted into the endogenous rfc gene of the animal or cell, the animal or cell will have a reduction or elimination in the expression and/or biological activity of RFC as described above. As another example, in one embodiment of the invention, a genetically modified mouse is produced by inserting into the genome of an embryonic stem (ES) cell an isolated nucleic acid molecule (e.g., a targeting vector) having an isolated nucleic acid sequence encoding the murine rfc gene. In this isolated nucleic acid sequence, exons 1-4 of the murine rfc gene has been deleted and replaced with a non-RFC nucleic acid sequence (e.g., a marker sequence, such as a neomycin cassette). The isolated nucleic acid molecule is preferably designed such that when the molecule is injected into embryonic stem (ES) cells, the isolated nucleic acid molecule will integrate into the genome of the cells, preferably at the endogenous rfc gene (i.e., targeted integration).

Techniques for achieving targeted integration of an isolated nucleic acid molecule into a genome are well known in the art and are described, for example in “Manipulating the Mouse embryo”, supra. For example, the isolated nucleic acid molecule can be engineered into a targeting vector which is designed to integrate into a host genome. According to the present invention, a targeting vector is defined as a nucleic acid molecule which has the following three features: (1) genomic sequence from the target gene in the host genome to stimulate homologous recombination at that gene; (2) a desired genetic modification within the genomic sequence from the target gene sufficient to obtain the desired phenotype; and (3) a selectable marker (e.g., an antibiotic resistance cassette, such as G418 (i.e., neomycin) or hygromycin resistance cassettes). Such targeting vectors are well known in the art. Following introduction of the isolated nucleic acid molecule of the targeting vector into the ES cells, ES cells which homologously integrate the isolated nucleic acid molecule are injected into mouse blastocysts and chimeric mice are produced. These mice are then bred onto the desired mouse background to detect those which transmit the mutated gene through the germline. Heterozygous offspring of germline transmitting lines can then be mated to produce homozygous progeny when these are viable (e.g., when another source of RFC is provided, such as by transfection of the human rfc according to the present invention), or to study developmental defects when they are not.

As yet another example, a genetically modified mouse is produced by nuclear transfer techniques. Briefly, in nuclear transfer, transfer of a blastomere nucleus from a somatic cell to the cytoplasm of an enucleated oocyte or zygote allows for the production of genetically identical individuals. To produce the genetically modified animal of the present invention, somatic cells are first genetically modified to have decreased or no expression of the endogenous RFC as described above, and in some embodiments, nucleic acids encoding the human RFC are introduced into the cell. The nuclei of the transfected primary cell lines are then used for nuclear transfer into donor enucleated oocytes or zygotes.

Mice which carry one or more mutated RFC alleles can be identified using any suitable method for evaluating DNA. For example, genotypes can be analyzed by PCR and confirmed by Southern blot analysis as described (Sambrook et el., 1988, Molecular Cloning: A Laboratory Manual, Cold Spring Harbor Press, Cold Spring Harbor Laboratory, Cold Spring Harbor, N.Y., or Current Protocols in Molecular Biology (1989) and supplements).

According to the present invention, an isolated nucleic acid molecule suitable for use in the present invention (e.g., suitable for use in a targeting vector or other molecular genetic technique according to the invention) is typically produced using recombinant DNA technology (e.g., polymerase chain reaction (PCR) amplification, cloning) or chemical synthesis. DNA comprising the designed nucleic acid sequence (e.g., the rfc gene, modified or unmodified) may be created, for example, by using polymerase chain reaction (PCR) techniques or other cloning techniques. The template can be a genomic or cDNA library. Such methodologies are well known in the art (Sambrook et al., supra).

In accordance with the present invention, an isolated nucleic acid molecule is a nucleic acid molecule that has been removed from its natural milieu (i.e., that has been subject to human manipulation), its natural milieu being the genome or chromosome in which the nucleic acid molecule is found in nature. As such, “isolated” does not necessarily reflect the extent to which the nucleic acid molecule has been purified, but indicates that the molecule does not include an entire genome or an entire chromosome in which the nucleic acid molecule is found in nature. An isolated nucleic acid molecule can include a gene. An isolated nucleic acid molecule that includes a gene is not a fragment of a chromosome that includes such gene, but rather includes the coding region and regulatory regions associated with the gene, but no additional genes naturally found on the same chromosome. An isolated nucleic acid molecule can also include a specified nucleic acid sequence flanked by (i.e., at the 5′ and/or the 3′ end of the sequence) additional nucleic acids that do not normally flank the specified nucleic acid sequence in nature (i.e., heterologous sequences). Isolated nucleic acid molecule can include DNA, RNA (e.g., mRNA), or derivatives of either DNA or RNA (e.g., cDNA). Although the phrase “nucleic acid molecule” primarily refers to the physical nucleic acid molecule and the phrase “nucleic acid sequence” primarily refers to the sequence of nucleotides on the nucleic acid molecule, the two phrases can be used interchangeably, especially with respect to a nucleic acid molecule, or a nucleic acid sequence, being capable of encoding a protein.

Isolated nucleic acid molecules useful in the present invention can be modified by nucleotide insertions, deletions, and substitutions (e.g., nucleic acid homologues) in a manner such that the modifications produce the desired effect (e.g., a deletion or substitution of a portion of a rfc gene sufficient to reduce RFC expression and/or biological activity in an animal when the nucleic acid molecule is integrated into the animal's genome). An isolated nucleic acid molecule encoding RFC can include degeneracies. As used herein, nucleotide degeneracies refers to the phenomenon that one amino acid can be encoded by different nucleotide codons. Thus, the nucleic acid sequence of a nucleic acid molecule that encodes a RFC of the present invention can vary due to degeneracies.

As used herein, a wild-type sibling, or wild-type littermate, of a non-human animal of the present invention, is an animal which is born to the same or genetically identical parents as a genetically modified animal described herein, and preferably, is born in the same litter as a genetically modified animal described herein, but which did not inherit a genetically modified allele at the rfc gene. Such an animal is essentially a normal animal and is useful as an age-matched control for the methods described herein.

One embodiment of the present invention relates to a method for studying the molecular and biochemical events associated with folate transport, and particularly with developmental effects and cancer therapy. Such a method can be performed in vitro (e.g., by using cells, tissues or body fluids of the genetically modified animal) or in vivo (e.g., by administering regulatory compounds to a genetically modified animal of the present invention and evaluating the effects of such compounds in vivo). Regulatory compounds identified by this method can be useful for treating deficiencies in folate transport, for treating or preventing developmental defects, or for treating a condition associated with impaired folate (or anti-folate) transport, such as cancer, cardiovascular disease, and neurodegenerative diseases (e.g., Alzheimer's, Parkinson's).

In one aspect, such a method includes the steps of: (a) harvesting cells, tissues or body fluids from a genetically modified non-human animal of the present invention; and, (b) comparing the cells, tissues or body fluids from the genetically modified non-human animal to cells, tissues or body fluids from a wild-type sibling of the genetically modified non-human animal or from another suitable control (e.g., a wild-type animal of the same species or strain that is not a sibling, a heterozygous mutant animal when the experimental animal is a homozygous mutant, etc.). The step of harvesting is performed using any of the well known methods of harvesting cells, tissues and/or body fluids from an animal, and depend on the tissues to be studied and the status of the experiment to be performed. For example, cells can be harvested by biopsy, dissection, or lavage; tissues can be harvested by surgery, biopsy or dissection; and body fluids can be harvested by withdrawal, swiping, or lavage.

The step of comparing is performed by an assay that is suitable for the tissue to be evaluated and the goal of the experiment. For example, suitable assays which might be performed on the cells, tissues, and/or body fluids of a genetically modified non-human animal of the present invention include, but are not limited to: morphological examination of the cells, tissues or body fluids, histological examination of the cells, tissues or body fluids; evaluation of RFC biological activity in the animal; evaluation of folate metabolism in the animal; evaluation of blood biochemistry in the animal. A variety of such assays are well known in the art.

Another embodiment of the present invention relates to a method to identify compounds, and particularly, folate homologues and mimetics, for use in treating cancer. Folate homologues and mimetics that are useful for treating cancer typically antagonize the biological activity of folate, and are therefore commonly referred to as anti-folates. Methotrexate is an example of a well-known anti-folate. Such a method includes screening a compound to be evaluated for its ability to inhibit growth of neoplasms in treating cancer. Specifically, the method of identifying anti-folate compounds for the treatment of cancer of the present invention includes the steps of: (a) contacting an anti-folate compound to be evaluated with cells of a genetically modified non-human animal having human reduced folate carrier (RFC) biological activity. The genome of the non-human animal comprises: (1) a first genetic modification in two alleles of its endogenous reduced folate carrier (rfc) gene, wherein the first genetic modification results in an absence of endogenous reduced folate carrier activity; and (2) a second genetic modification that results in expression by the cells of the animal of a nucleic acid molecule comprising a nucleic acid sequence encoding a biologically active human reduced folate carrier (RFC). The method further includes the step (b) of selecting compounds from step (a) that bind to the human RFC expressed by the cells.

In a further, or alternate aspect of this embodiment of the invention, the method can include a step of evaluating physiological and pathological changes in the genetically modified non-human animal as compared to a non-human animal selected from the group of: (i) a second genetically modified non-human animal comprising a genetic modification within at least one allele of its rfc gene, wherein the genetic modification results in a reduction in expression and/or biological activity in the animal; and/or (ii) a third non-human animal having a genome comprising a wild-type rfc gene at two (i.e., both) alleles.

The step of contacting the RFC with a compound can occur in vivo or in vitro. When the step is in vivo, the compound to be evaluated is administered to the non-human animal by any suitable route of administration (discussed in detail below). In this aspect, the step of selecting typically comprises harvesting a cell source from the non-human animal selected from the group consisting of a cell sample, a tissue or a body fluid, and measuring binding of the compound to the RFC and/or measuring cellular uptake of the compound by the cells. In one embodiment, in vivo administration of the compound can be useful, for example, when the non-human animal has been induced to grow tumor cells, and wherein the method further comprises a step of selecting compounds from (a) that, when administered to the animal, result in a reduction in growth of the tumor cells.

Alternatively, the step of contacting the compound with the RFC can be performed in vitro. Such a method typically comprises harvesting a cell source selected from the group consisting of a cell sample, a tissue or a body fluid from the non-human animal first, followed by contacting cells in the cell source with the compound in vitro. In this embodiment, the step of selecting can again include selecting compounds from (a) that bind the human RFC (e.g., by performing a binding assay) and/or by measuring cellular uptake of the compound into the cell expressing the human RFC.

In one embodiment, compounds are selected that bind to the human RFC with a greater affinity than a control compound that binds to human RFC. Such compounds may be more efficiently transported into the cell, which may have a therapeutic benefit, particularly in view of the problems associated with resistance to some common anti-folates (e.g., methotrexate) that is frequently observed in cancer patients. In this regard, the control compound that binds to human RFC can be any known compound, including the natural ligand, folate, but may also be a compound such as methotrexate, particularly when the goal of the evaluation is to select a compound that binds to the RFC with a higher affinity than methotrexate.

In another aspect, the cells of the non-human animal are engineered to express the human reduced folate carrier at a level that is less than the level of expression expected for expression of endogenous RFC in a wild-type animal. As discussed previously herein, it is known in the art that one mechanism that is responsible for impaired anti-folate response in some human patients is decreased, or impaired, expression of the RFC. Therefore, one can construct a model of such decreased expression by controlling the expression level of the human RFC in the non-human animal to be less than that observed for the endogenous RFC in the wild-type animal, or alternatively, to mimic an expression level that is typically observed in patients with this impairment. Therefore, compounds can be selected that bind to and are effectively transported by the RFC, particularly when the expression level of the RFC is low. Such compounds may be more effective at lower doses, may have higher affinity for the RFC, or may have some other characteristic that allows the compound to be efficiently transported into the cell despite the low number of available transporters on the cell.

In yet another aspect, the human RFC expressed by the non-human animal is a mutant human RFC that is known to have reduced binding affinity or reduced transport ability for at least one known anti-folate compound. As discussed previously, some patients with resistance to known anti-folates have been found to have mutated or impaired RFC proteins. Once the nucleic acid sequence encoding such mutant or impaired RFCs is determined, such nucleic acid sequences for mutant RFCs can be expressed in a non-human animal of the present invention and used to select anti-folate compounds that bind to and are transported into a cell expressing the RFC with a suitable efficiency.

In one embodiment of the present invention, the genetically modified non-human animal that expresses a human RFC can be useful for evaluating anti-folate compounds for the treatment of cancer that enter the cell by a mechanism that does not include binding to the RFC. Such a method would be useful for identifying anti-folates for use in patients with impaired RFC expression or transport activity, since such compounds do not rely on the RFC to enter the cell. Such a method would include the steps of: (a) contacting an anti-folate compound to be evaluated with cells of a genetically modified non-human animal having human reduced folate carrier (RFC) biological activity as described above; and (b) selecting compounds from (a) that do not bind to the human RFC expressed by the cells, but which are internalized by the cells. The basic steps of the method can be performed in a similar manner as the method for identifying compounds that do bind to the RFC except that the endpoint of the assay would be to confirm non-binding to the RFC, but internalization (uptake) of the compound into the cells.

Any compounds identified using the method of the present invention are candidates for use in the treatment of a patient with cancer. Such compounds can then be administered to a patient with cancer to provide a therapeutic benefit to such patient.

Yet another embodiment of the present invention relates to a method to identify compounds useful for treating a condition associated with defective folate transport in an animal. Such a method includes the steps of: (a) contacting a compound to be evaluated with cells of a genetically modified non-human animal that has a genome comprising a genetic modification in at least one allele of the endogenous reduced folate carrier (rfc) gene in the animal, wherein the genetic modification results in a reduction in endogenous reduced folate carrier (RFC) activity in the animal; and, (b) selecting compounds from (a) that bind to the RFC expressed by the cells. In a preferred embodiment, the genome of the animal comprises a first genetic modification in two alleles of its endogenous reduced folate carrier (rfc) gene, wherein the first genetic modification results in an absence of endogenous reduced folate carrier activity, and a second genetic modification that results in expression by cells in the animal of a nucleic acid molecule comprising a nucleic acid sequence encoding a biologically active human reduced folate carrier (RFC).

In this embodiment of the invention, the step of selecting can include selecting compounds that bind to the RFC with a higher affinity than the affinity of folate for the RFC. Such compounds may be suitable folate agonists which could be used at lower doses than folate, for example. In another aspect, the human RFC can be a mutant human RFC that is known to be associated with the presence of a condition associated with defective folate transport in an animal. Such a method can include evaluating folate analogs for the ability to bind to the mutant human RFC with an affinity that is greater than the affinity of folate for the mutant human RFC, for example.

Conditions for which compounds identified by this method may be therapeutically useful include, but are not limited to, a neural tube defect, Parkinson's disease, Alzheimer's disease, a cardiovascular disease, stroke, or cleft palate. The present invention contemplates the use of any compounds identified by the present method to treat any of such conditions, preferably by administration of such compounds to the patient.

Each of the above-described methods of identification of folate and anti-folate compounds for use in the treatment of cancer or conditions associated with defective folate transport uses a genetically modified non-human animal of the present invention, such animals being described in detail above. In addition, the steps of contacting and selecting for each method are described in detail below. As discussed above, the step of contacting can be performed in vivo or in vitro; administration and dosing protocols are described in detail below.

The present methods involve contacting cells that express a reduced folate carrier with the compound being tested for a sufficient time to allow for interaction of the compound with the RFC, and in some embodiments, transport of the compound by the RFC into the cell. The period of contact with the compound being tested can be varied depending on the result being measured (e.g., binding alone versus binding and transport), and can be determined by one of skill in the art. For example, for binding assays, a shorter time of contact with the compound being tested is typically suitable, than when transport is assessed. As used herein, the term “contact period” refers to the time period during which the cells expressing an RFC are in contact with the compound being tested.

In one embodiment, the conditions under which a receptor according to the present invention is contacted with a compound to be evaluated, such as by mixing the compound with cells expressing the RFC (in vitro), or by administering the compound to the animal in a manner effective to deliver the compound to the cells expressing the RFC (in vivo), are conditions in which the RFC is not bound (e.g., essentially no folate or anti-folate compound is present or expected to be present). For example, such conditions include normal culture conditions in the absence of a folate or anti-folate compound, or conditions under which the non-human animal has not received an exogenous source of a folate compound for a time sufficient to substantially reduce or eliminate the presence of folate in the system of the animal (e.g., in its diet). In this embodiment, the compound to be evaluated is then contacted with the receptor (e.g., by administration or by in vitro mixing).

The step of selecting compounds that bind to or are transported by the RFC into the cell can be performed by any standard binding assay or measurement of cellular uptake of the compound, respectively. Binding assays are well known in the art and include, but are not limited to, competitive binding techniques, equilibrium dialysis or BIAcore methods. Cellular uptake assays are also well known in the art and include measurement of labeled compound, for example. Suitable controls can be used, as appropriate (i.e., a known folate or anti-folate, or a negative control, labeled or unlabeled, depending on the assay).

According to the present invention, suitable compounds to be evaluated as folate or anti-folate analogs in the present method preferably include compounds which have an unknown regulatory activity or an undetermined level of activity, at least with respect to the ability of such compounds to bind to and be transported by the reduced folate carrier, and to act as a folate analog or an anti-folate analog. Particularly preferred putative regulatory compounds to test in the method of the present invention include any agonist or antagonist (also referred to as an anti-folate) of folate biological activity, and can include a homologue of folate with folate agonist or antagonist activity, a peptide or non-peptide mimetic of folate with folate agonist or antagonist activity, or a fusion protein including a folate agonist or antagonist peptide. A suitable regulatory compound can also include any compound, such as a small molecule or drug that has folate agonist or antagonist activity (i.e., it need not necessarily be structurally similar to folate).

According to the present invention, the term “compound” encompasses any of the following compounds: a peptide, a fragment of a known peptide (including both biologically active and inactive fragments), a homologue of such a peptide, a mimetic (peptide or non-peptide) of such a peptide, a fusion protein comprising such a peptide, and any pharmaceutical salts of such a peptide, as well as any small molecule or drug. In addition, peptides useful as compounds to be evaluated in the present invention may exist, particularly when formulated, as dimers, trimers, tetramers, and other multimers. Such multimers are included within the scope of the present invention. As used herein, the term “analog”, as used in connection with a folate or anti-folate compound according to the present invention, refers generically to any homologue or mimetic (peptide or non-peptide) of folate. Homologues and mimetics are described in detail below. Analogs can include both agonists and antagonists of the natural RFC ligand, folate.

As used herein, the phrase “folate agonist” refers to any compound that interacts with a reduced folate carrier and that elicits an observable response. More particularly, a folate agonist can include, but is not limited to, a protein, a peptide, a nucleic acid, an antibody or antigen-binding fragment thereof, a carbohydrate-based compound, a lipid-based compound, a natural organic compound, a synthetically derived organic compound, or other compound (e.g., any product of drug design) that selectively binds to and/or is transported into a cell by a reduced folate carrier expressed by the cell, and most commonly includes a homologue or mimetic of folate, including a synthetic folate compound which is characterized by its ability to agonize (e.g., stimulate, induce, increase, enhance) the biological activity of a naturally occurring folate receptor (including the reduced folate carrier) in a manner similar to the natural agonist, folate.

The phrase, “folate antagonist” or “anti-folate” refers to any compound which inhibits the biological activity of a folate agonist, as described above. More particularly, a folate antagonist is capable of associating with receptors that bind to the natural ligand, folate, and may bind to these receptors such that the biological activity of the receptor is decreased (e.g., reduced, inhibited, blocked, reversed, altered) in a manner that is antagonistic (e.g., against, a reversal of, contrary to) to the action of the natural agonist, folate, on the receptor. According to the present invention, a preferred anti-folate may be an antagonist of folate biological activity, but may bind to the reduced folate carrier in a manner equivalent to or with greater affinity or avidity than the natural ligand folate. Such compounds may be folate antagonists, but not with regard to the ability of the compound to bind to and be transported by the reduced folate carrier. Such a compound can include, but is not limited to, a protein, a peptide, a nucleic acid, an antibody or antigen-binding fragment thereof, a carbohydrate-based compound, a lipid-based compound, a natural organic compound, a synthetically derived organic compound, or other compound (e.g., any product of drug design) that antagonizes the biological activity of folate, but which may bind to and be transported by the reduced folate carrier.

As used herein, the term “homologue” is used to refer to a peptide which differs from a naturally occurring peptide (i.e., the “prototype”) by minor modifications to the naturally occurring peptide, but which maintains the basic peptide and side chain structure of the naturally occurring form. Such changes include, but are not limited to: changes in one or a few amino acid side chains; changes one or a few amino acids, including deletions (e.g., a truncated version of the peptide) insertions and/or substitutions; changes in stereochemistry of one or a few atoms; and/or minor derivatizations, including but not limited to: methylation, glycosylation, phosphorylation, acetylation, myristoylation, prenylation, palmitation, amidation and/or addition of glycosylphosphatidyl inositol. Preferably, a homologue has either enhanced or substantially similar properties compared to the naturally occurring peptide as discussed above (i.e., agonists), although peptides with properties that antagonize the activity of the natural peptide (i.e., antagonists) are also encompassed by certain embodiments of the present invention.

Homologues can be the result of natural allelic variation or natural mutation, or they can be intentionally derived or produced. A naturally occurring allelic variant of a nucleic acid encoding a given protein is a gene that occurs at essentially the same locus (or loci) in the genome as the gene which encodes the protein, but which, due to natural variations caused by, for example, mutation or recombination, has a similar but not identical sequence. Allelic variants typically encode proteins having similar activity to that of the protein encoded by the gene to which they are being compared. One class of allelic variants can encode the same protein but have different nucleic acid sequences due to the degeneracy of the genetic code. Allelic variants can also comprise alterations in the 5′ or 3′ untranslated regions of the gene (e.g., in regulatory control regions). Allelic variants are well known to those skilled in the art.

Homologues can be produced using techniques known in the art for the production of proteins including, but not limited to, direct modifications to the isolated, naturally occurring protein, direct protein synthesis, or modifications to the nucleic acid sequence encoding the protein using, for example, classic or recombinant DNA techniques to effect random or targeted mutagenesis.

A mimetic refers to any peptide or non-peptide compound that is able to mimic the biological action of a naturally occurring peptide, often because the mimetic has a basic structure that mimics the basic structure of the naturally occurring peptide and/or has the salient biological properties of the naturally occurring peptide. Mimetics can include, but are not limited to: peptides that have substantial modifications from the prototype such as no side chain similarity with the naturally occurring peptide (such modifications, for example, may decrease its susceptibility to degradation); anti-idiotypic and/or catalytic antibodies, or fragments thereof, non-proteinaceous portions of an isolated protein (e.g., carbohydrate structures); or synthetic or natural organic molecules, including nucleic acids and drugs identified through combinatorial chemistry, for example. Such mimetics can be designed, selected and/or otherwise identified using a variety of methods known in the art. Various methods of drug design, useful to design mimetics or other therapeutic compounds useful in the present invention are disclosed in Maulik et al., 1997, Molecular Biotechnology: Therapeutic Applications and Strategies, Wiley-Liss, Inc., which is incorporated herein by reference in its entirety.

A folate mimetic can be obtained, for example, from molecular diversity strategies (a combination of related strategies allowing the rapid construction of large, chemically diverse molecule libraries), libraries of natural or synthetic compounds, in particular from chemical or combinatorial libraries (i.e., libraries of compounds that differ in sequence or size but that have the similar building blocks) or by rational, directed or random drug design. See for example, Maulik et al., supra.

In a molecular diversity strategy, large compound libraries are synthesized, for example, from peptides, oligonucleotides, carbohydrates and/or synthetic organic molecules, using biological, enzymatic and/or chemical approaches. The critical parameters in developing a molecular diversity strategy include subunit diversity, molecular size, and library diversity. The general goal of screening such libraries is to utilize sequential application of combinatorial selection to obtain high-affinity ligands for a desired target, and then to optimize the lead molecules by either random or directed design strategies. Methods of molecular diversity are described in detail in Maulik, et al., ibid.

Maulik et al. also disclose, for example, methods of directed design, in which the user directs the process of creating novel molecules from a fragment library of appropriately selected fragments; random design, in which the user uses a genetic or other algorithm to randomly mutate fragments and their combinations while simultaneously applying a selection criterion to evaluate the fitness of candidate ligands; and a grid-based approach in which the user calculates the interaction energy between three dimensional receptor structures and small fragment probes, followed by linking together of favorable probe sites.

According to the present invention, an isolated or biologically pure protein, including peptides and analogs thereof, is a protein that has been removed from its natural milieu. As such, “isolated” and “biologically pure” do not necessarily reflect the extent to which the protein has been purified. An isolated protein of the present invention can be obtained from its natural source, can be produced using recombinant DNA technology or can be produced by chemical synthesis. It is to be noted that the term “a” or “an” entity refers to one or more of that entity; for example, a compound refers to one or more compounds or at least one compound. As such, the terms “a” (or “an”), “one or more” and “at least one” can be used interchangeably herein. It is also to be noted that the terms “comprising”, “including”, and “having” can be used interchangeably. Furthermore, a compound “selected from the group consisting of” refers to one or more of the compounds in the list that follows, including mixtures (i.e., combinations) of two or more of the compounds.

In the practice of the methods of the present invention, when the compound to be evaluated is to be administered to the genetically modified non-human animal of the present invention, or when a selected compound is to be administered to a patient, it is useful, although not essential, to prepare formulations comprising an amount of the compound, either alone or in combination with a pharmaceutically acceptable salt and/or complexed with another suitable carrier (described below). Such formulations can be formulated for any route of administration, including, but not limited to, parenteral administration and transdermal administration. For example, formulations to be evaluated can be formulated in an excipient that the animal to be treated can tolerate. Examples of such excipients include water, saline, phosphate buffered solutions, Ringer's solution, dextrose solution, Hank's solution, polyethylene glycol-containing physiologically balanced salt solutions, and other aqueous physiologically balanced salt solutions. Nonaqueous vehicles, such as fixed oils, sesame oil, ethyl oleate, or triglycerides may also be used.

The formulations comprising one or more desired compounds typically contain from about 0.1% to 90% by weight of the active compound, preferably in a soluble form, and more generally from about 0.1% to 1.0%.

In one embodiment of the present invention, a pharmaceutically acceptable carrier can include additional compounds that increase the half-life of a formulation in the animal to which the compound is administered. Suitable carriers include, but are not limited to, polymeric controlled release vehicles, biodegradable implants, liposomes, bacteria, viruses, other cells, oils, esters, and glycols.

In one embodiment of the present invention, a formulation can include a controlled release composition that is capable of slowly releasing the formulation into an animal. As used herein, a controlled release composition comprises a compound to be evaluated or a compound that has been selected for use in a therapeutic composition in a controlled release vehicle. Suitable controlled release vehicles and devices include, but are not limited to, biocompatible polymers, polymeric matrices, capsules, microcapsules, microparticles, bolus preparations, osmotic pumps, diffusion devices, liposomes, lipospheres, and transdermal delivery systems. Other controlled release compositions of the present invention include liquids that, upon administration to an animal, form a solid or a gel in situ. Preferred controlled release compositions are biodegradable (i.e., bioerodible).

According to the present invention, an effective administration protocol (i.e., administering a compound to be evaluated, a therapeutic compound, or a formulation comprising such a compound in an effective manner) comprises suitable dose parameters and modes of administration that are not toxic to the animal, and which would reasonably be expected to be capable of binding to and being transported by the reduced folate carrier in the animal. When the compound is used as a therapeutic, the compound is administered by a protocol that is effective to provide a measurable change in the condition which the compound is intended to treat when administered one or more times over a suitable time period. It is well within the ability of one of skill in the art to establish a suitable dose and administration protocol for evaluating the ability of a compound to bind to and be transported by an RFC in a genetically modified non-human animal of the present invention, or to determine an effective dose of a selected compound to be administered to a patient for treatment. Effective dose parameters can be determined using methods standard in the art for a particular animal and condition. Such methods include, for example, determination of survival rates, side effects (i.e., toxicity) and other health factors associated with the administration of the compound.

Modes of administration of a compound or formulation of the present invention include any method of administration which results in delivery of the composition to the animal and particularly, to the reduced folate carrier expressed by cells in the animal. Such modes of administration can include, but are not limited to, oral, nasal, topical, transdermal, rectal, and parenteral routes. Parenteral routes can include, but are not limited to subcutaneous, intradermal, intravenous, intraperitoneal and intramuscular routes.

According to the present invention, to “treat” a disorder, such as a disorder associated with defective folate transport (e.g., neural tube defects) refers to reducing or ameliorating the disorder in a patient that suffers from the disorder, and to “prevent” a disorder refers to halting the disorder in a patient that is at risk of suffering from the disorder before the disorder becomes overt or develops at all. Preferably, the disorder, or the potential for developing the disorder, is reduced, optimally, to an extent that the patient no longer suffers from or does not develop the disorder, or the discomfort and/or altered functions and detrimental conditions associated with such disorder.

In accordance with the present invention, a suitable or effective single dose size is a dose that is capable of binding to a reduced folate carrier and in therapeutic embodiments, of causing a measurable change in a condition to be treated when administered one or more times over a suitable time period. Doses can vary depending upon the condition of the patient being treated, including the severity of the condition and/or any other related or non-related health factors experienced by a particular patient. Typically, the method of the present invention comprises administering a compound in a dose between about 0.1 μg and about 100 mg per kilogram body weight of the patient, and preferably, between about 0.1 μg and about 10 mg per kilogram body weight of the patient, and more preferably, between about 0.1 μg and about 1 μg per kilogram body weight of the patient, and even more preferably, between about 1 μg and about 10 mg per kilogram body weight of the patient.

In the therapeutic methods of the present invention, a therapeutic compound, including folate agonists and anti-folates, can be administered to any organism, and particularly, to any member of the Vertebrate class, Mammalia, including, without limitation, primates, rodents, livestock and domestic pets. Preferred mammals to treat include humans.

Various aspects of the present invention are illustrated in the following examples, which are provided for the purposes of illustration and are not intended to limit the scope of the present invention.

### EXAMPLES

### Example 1

This example describes the production of a heterozygous mutant RFC mouse of the present invention, wherein one allele of the endogenous rfc gene has been deleted.

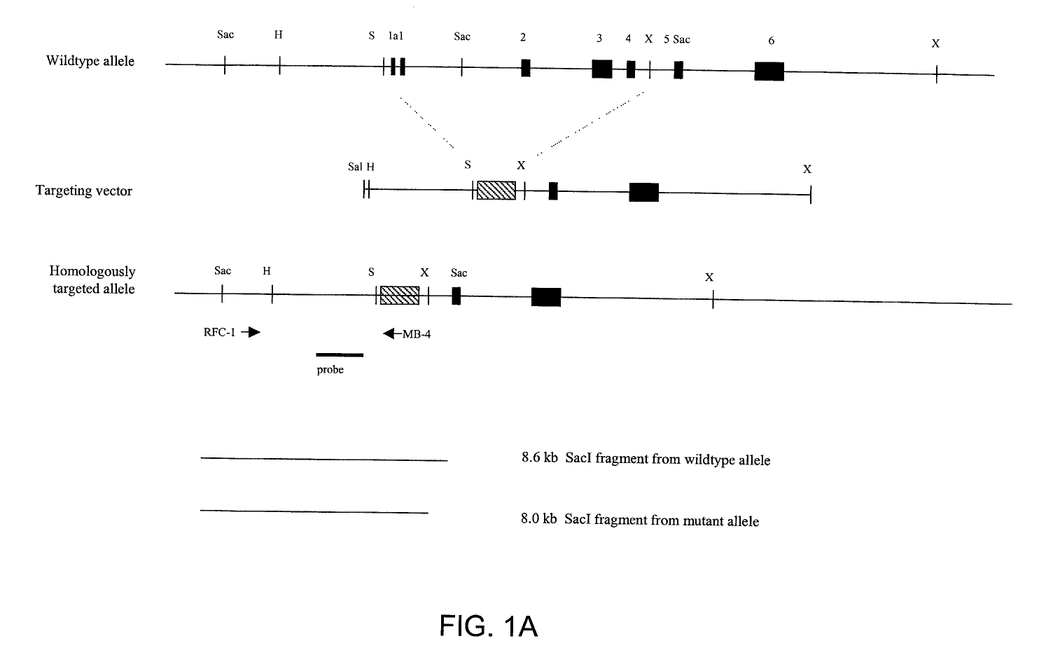

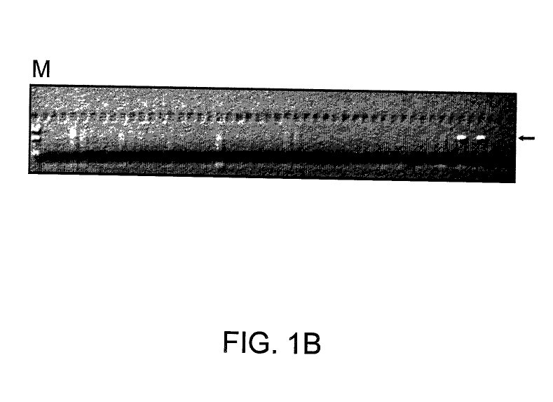

A mouse genomic DNA clone was isolated and modified by deletion of exons 1-4 and substitution with a G418 resistance cassette. FIG. 1A is a schematic diagram of the murine RFC-1 locus, the targeting vector, and the rearranged locus after homologous targeting. Exons are depicted as black boxes (1a, 1, 2, 3, 4, 5, and 6). Restriction sites are marked as necessary: Sac, SacI; H, HindIII; S, SmaI; X, XbaI; Sal, SalI. The targeting vector consists of a 3.4 kb HindIII-SmaI fragment 5′ of the neo cassette, and a 10 kb XbaI fragment, containing exons 5 and 6 of the rfc gene, 3′ of the neo cassette. Homologous integration of the cassette causes deletion of a 9 kb fragment, spanning exons 1a through 4. Briefly, the targeting vector was linearized by SalI and electroporated into Ab2.2 Embryonic Stem (ES) cells (Lexicon Genetics), following standard procedures. Individual clones were picked after 2 weeks of G418 selection (i.e., transfectants were selected for G418 resistance). Master plates were frozen and replica plates were processed for DNA.

DNAs were isolated from G418 resistant transfectants and analyzed by polymerase chain reaction to identify transfectants which had integrated the modified rfc DNA by homologous recombination at one of the endogenous rfc loci. Briefly, DNA was prepared from G418-resistant ES cell clones following standard procedures. One microliter of ES cell DNA was transferred to “Expand Long Template PCR” (Boehringer/Roche) reactions containing primers RFC-1 (5′TGCCA GGTGA CC AGCATCCATTGT3′) (SEQ ID NO:9) and MB-4 (5′GGGGA CTTTCCACACCCTAACTGA3′) (SEQ ID NO: 10). PCR reactions were pipetted and carried out according to the manufacturer's recommendations. FIG. 1B is an ethidium bromide stain of a gel running PCR reactions with embryonic stem cell DNA (ES) as templates and primers RFC-1 and MB-4. Primer RFC-1 is located 5′ of the integration site of the targeting vector, and primer MB-4 is located at the 5′ end of the neo cassette. Homologous integration of the targeting vector results in amplification of a 3.5 kb PCR fragment.

Embryonic stem cell clones with integration of the modified rfc gene at one of the endogenous rfc loci were further checked by Southern blot analysis to ensure that only one modified rfc gene was integrated into the genome of the mouse embryonic stem cell transfectants. Cells from a mouse embryonic stem cell line with one integration of the modified rfc gene at one of the endogenous rfc loci were injected into the blastoceols of mouse blastocysts. These manipulated mouse blastocysts were reimplanted into the uterine horns of pseudopregnant female mice. The progeny mice were analyzed for chimerism: as the embryonic stem cell lines are agouti while the recipient blastocysts are black, chimeric offspring are a mixture of black and agouti coat color.

Chimeric mice were then tested for the transmission of the modified rfc allele through the germline. Specifically, the chimeric mice were mated and the progeny analyzed for the presence of the modified rfc allele by polymerase chain reaction as described above. The modified rfc allele was transmitted to the progeny. These progeny were heterozygous at the rfc locus: they had one wild-type rfc allele and one modified rfc allele. When these heterozygotes were mated no progeny homozygous for the modified rfc allele were born alive. This result indicates that mouse mutants homozygous for the modified rfc gene die in utero.

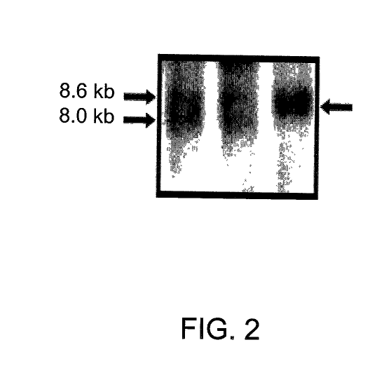

FIG. 2 is a digitized image of a Southern blot showing SacI-digested DNA from heterozygous and wildtype pups. Briefly, high molecular weight DNA was prepared from brains of 1 week old pups from matings of heterozygous carriers. The DNA was digested with SacI, electrophoresed in a 1% agarose gel, and blotted to a Nylon membrane according to standard procedures. A radioactively labeled probe was generated by nick-translation of a 1.5 kb PCR fragment using primers RFC-4 (5′TATAGACTGCACCAGCTGTG CACG3′) (SEQ ID NO:11) and RFC-5 (5′AAATTGACCATGCCGGCCATACGG3′) (SEQ ID NO:12). These primers are located within the HindIII-SmaI fragment of the targeting vector. The hybridized membrane was exposed to XAR film for 4 days. Referring to FIG. 2, the probe fragment is located within the 3.4 kb HindIII-SmaI fragment of the targeting vector. Hybridization of this probe to SacI-digested DNA lights up a wildtype 8.6 kb fragment spanning from a SacI site 5′ of the HindIII site which starts the targeting construct and a SacI site in intron 1. The homologously targeted allele produces a slightly shorter 8.0 kb fragment spanning from the same 5′ SacI site as in the wildtype allele and a 3′ SacI site within exon 5 (since the SacI site in intron 1 is deleted).

### Example 2

This example describes the production of a mouse mutant that carries and expresses the human rfc gene.

A human genomic DNA sequence including the rfc gene was isolated in a P1/BAC vector. The clone was tested for presence of the 5′ and 3′ ends of the rfc gene by the polymerase chain reaction. Fertilized mouse oocytes were isolated and human rfc DNA was injected into the male pronucleus. The manipulated oocytes were reimplanted into pseudo-pregnant females. DNAs from progeny born to these females were analyzed both for the presence of the human rfc gene, by the polymerase chain reaction, and for expression of the human rfc gene by reverse transcriptase-polymerase chain reaction.

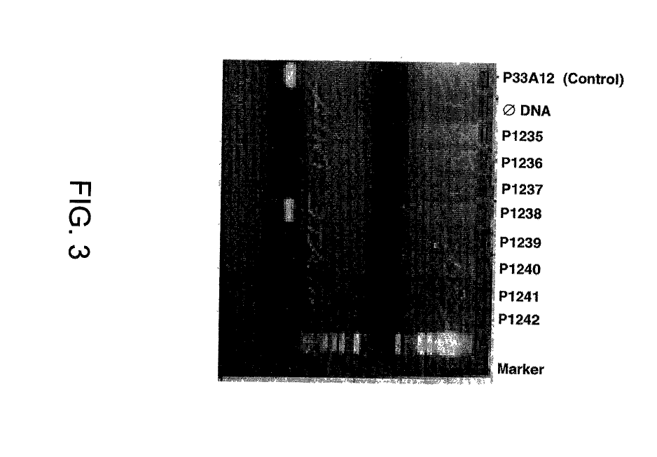

FIG. 3 is a scan of an agarose gel electrophotogram of PCR products from tail DNAs of transgenic mice. The primers are specific for the human rfc gene demonstrating integration of the human rfc gene in the mouse germline.

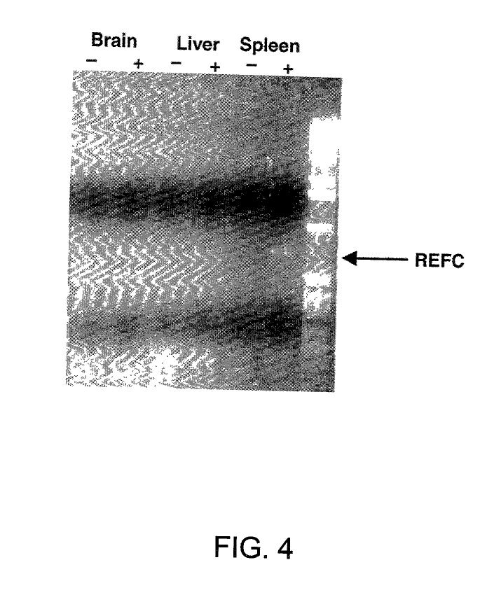

FIG. 4 is a digitized image of a gel showing RT-PCR products from RNA of an RFC transgenic mouse amplified with human specific RFC primers. Brain, liver and spleen were harvested from a human RFC transgenic mouse, and RNAs were isolated. These RNAs were used as templates in RT-PCR reactions with human specific RFC primers. All three tissues express the human RFC transgene.

### Example 3

This example shows that the reduced folate carrier plays a role in neural development.

Methods

Whole-mount in situ Hybridization

Whole mount in situ hybridizations of embryos were performed as described by Cash et al., 1997, J. Cell Biol. 136:445-457). For riboprobes, a 1.1 kb fragment of the mouse rfc was amplified by PCR from an E8.5 mouse cDNA library, cloned, and used to make digoxigenin labeled sense and anti-sense RNA. Probes were detected using a sheep anti-digoxigenin conjugated alkaline phosphatase antibody (Boehringer Mannheim).

For the preparation of sections, whole-mount embryos were re-fixed in 10% formaldehyde, and processed for paraffin-embedding. Sections were cut at 16-20 μM and mounted.

Northern Analysis

Embryos were collected at E9.5 and divided by a mid-line cut made slightly caudal to the first brachial arch, and a second cut more caudal so as to include most of the peritoneal gut. The segments of embryos (˜8) were pooled, homogenized in TRIzol reagent (Gibco/BRL), and RNA extracted. RNA was fractionated on a formaldehyde-agarose gel, transferred to nylon membrane, and hybridized (Sambrook, 1989, Molecular Cloning: A Laboratory Manual, Cold Spring Harbor Press, Cold Spring Harbor Laboratory, Cold Spring Harbor, N.Y.) with radiolabeled probes.

Reverse-Transcriptase Polymerase Chain Reaction

Embryonic RNA isolated as above was used to synthesize the first strand cDNA (Murray et al., 1996, J. Biol. Chem. 271:19174-19179) which subsequently served as a PCR template for amplification of a 338 bp fragment of the mouse rfc cDNA.

Results

To investigate the requirement for folates during development, the inventors investigated the pattern of rfc expression during embryogenesis. Prior to embryonic (E) day 7.75, expression of rfc RNA was weak, but from E7.75 through E12.5, there was a low-level generalized expression, as well as regions of highly specific staining. Bands flanking the neural tube (NT) moved caudally during embryogenesis (not shown) and after E10.5, specific expression was restricted to a single stripe between the last caudal somite and the tail bud. Lateral sections through the embryo tail at E9.5 suggested that this rfc expression was highest at the newly-formed edge of the last somite, while transverse sections indicated that the rfc mRNA was primarily restricted to a layer subadjacent to the ectoderm within the dorsal aspect of the forming somite (not shown).

Northern analysis indicated that the rfc message was produced in equal abundance throughout the embryo, and this was confirmed by cloning and sequencing of RT-PCR products.

The enhanced expression of the rfc mRNA in the presomitic tissue and head regions suggests a role for folate transport during neurogenesis.

While various embodiments of the present invention have been described in detail, it is apparent that modifications and adaptations of those embodiments will occur to those skilled in the art. It is to be expressly understood, however, that such modifications and adaptations are within the scope of the present invention.

What is claimed is:

1. A genetically modified non-human animal comprising a genetic modification in at least one allele of the endogenous reduced folate carrier (rfc) gene in said animal, wherein said genetic modification results in a reduction in endogenous reduced folate carrier (RFC) expression or biological activity in said animal.

2. The genetically modified non-human animal of claim 1, wherein said genetic modification is in both alleles of the endogenous rfc gene in said animal.

3. The genetically modified non-human animal of claim 1, wherein said genetic modification is a modification of nucleotides in a gene encoding reduced folate carrier (RFC) in said animal selected from the group consisting of a deletion of nucleotides from said gene, an insertion of nucleotides in said gene, a substitution of nucleotides for nucleotides in said gene, and an inversion of nucleotides in said gene.

4. The genetically modified non-human animal of claim 1, wherein said genetic modification comprises a modification within exons 1-4 of the rfc gene in said animal.

5. The genetically modified non-human animal of claim 1, wherein said genetic modification comprises a modification with a regulatory region of the rfc gene in said animal.

6. The genetically modified non-human animal of claim 1, wherein said genetic modification is a deletion of a nucleic acid sequence within at least one allele of the rfc gene in said animal.

7. The genetically modified non-human animal of claim 1, wherein said genetic modification is a deletion of both alleles of the rfc gene in said animal, wherein the genetic modification results in an absence of endogenous reduced folate carrier (RFC) biological activity in the animal.

8. The genetically modified non-human animal of claim 1, wherein said genetic modification is a deletion of at least a portion of a nucleic acid sequence comprising exons 1-4 of the gene encoding RFC.

9. The genetically modified non-human animal of claim 1, wherein said non-human animal is a mouse.

10. The genetically modified non-human animal of claim 11, wherein said genetic modification is a deletion from the genome of said mouse of a nucleic acid sequence comprising SEQ ID NO: 1.

11. A genetically modified non-human animal having human reduced folate carrier (RFC) biological activity, wherein said animal comprises a first genetic modification in two alleles of an endogenous reduced folate carrier (rfc) gene, wherein said first genetic modification results in an absence of endogenous reduced folate carrier expression or biological activity, and wherein said animal further comprises a second genetic modification that results in expression by cells in said animal of a nucleic acid molecule comprising a nucleic acid sequence encoding a biologically active human reduced folate carrier (RFC).

12. The genetically modified non-human animal of claim 11, wherein said first genetic modification in said endogenous reduced folate carrier (rfc) gene is selected from the group consisting of: a deletion of nucleotides from said gene, an insertion of nucleotides in said gene, a substitution of nucleotides for nucleotides in said gene, and an inversion of nucleotides in said gene.

13. The genetically modified non-human animal of claim 11, wherein said first genetic modification in said endogenous reduced folate carrier (rfc) gene comprises a modification within exons 1-4 of said rfc gene.

14. The genetically modified non-human animal of claim 11, wherein said first genetic modification in said endogenous reduced folate carrier (rfc) gene comprises a modification with a regulatory region of said rfc gene.

15. The genetically modified non-human animal of claim 11, wherein said first genetic modification in said endogenous reduced folate carrier (rfc) gene is a deletion of both alleles of said rfc gene.

16. The genetically modified non-human animal of claim 11, wherein said first genetic modification in said endogenous reduced folate carrier (rfc) gene is a deletion of at least a portion of a nucleic acid sequence comprising exons 1-4 of the gene encoding reduced folate carrier (RFC).

17. The genetically modified non-human animal of claim 11, wherein said nucleic acid sequence encoding human reduced folate carrier comprises SEQ ID NO:3.

18. The genetically modified non-human animal of claim 11, wherein said nucleic acid sequence encoding human reduced folate carrier is operatively linked to a human transcriptional control sequence.

19. The genetically modified non-human animal of claim 11, wherein said nucleic acid sequence encoding human reduced folate carrier is operatively linked to a transcriptional control sequence for said endogenous rfc gene.

20. The genetically modified non-human animal of claim 11, wherein the cells of said non-human animal express said human reduced folate carrier at a level that is equivalent to the level of expression expected for expression of endogenous RFC in a wild-type animal.

21. The genetically modified non-human animal of claim 11, wherein the cells of said non-human animal express said human reduced folate carrier at a level that is less than the level of expression expected for expression of endogenous RFC in a wild-type animal.

22. The genetically modified non-human animal of claim 11, wherein the cells of said non-human animal express said human reduced folate carrier at a level that is greater than the level of expression expected for expression of endogenous RFC in a wild-type animal.

23. The genetically modified non-human animal of claim 11, wherein said non-human animal is a mouse.

24. The genetically modified non-human animal of claim 23, wherein said genetic modification in said endogenous reduced folate carrier (rfc) gene is a deletion from the genome of said mouse of a nucleic acid sequence comprising SEQ ID NO: 1.

25. The genetically modified non-human animal of claim 23, wherein said nucleic acid sequence encoding human reduced folate carrier comprises SEQ ID NO:3.

26. A method to evaluate anti-folate compounds for the treatment of cancer, comprising:
a. contacting an anti-folate compound to be evaluated with cells of a genetically modified non-human animal having human reduced folate carrier (RFC) biological activity, wherein the genome of said non-human animal comprises a first genetic modification in two alleles of its endogenous reduced folate carrier (rfc) gene, wherein said first genetic modification results in an absence of endogenous reduced folate carrier activity, and wherein the genome of said animal further comprises a second genetic modification that results in expression by said cells of said animal of a nucleic acid molecule comprising a nucleic acid sequence encoding a biologically active human reduced folate carrier (RFC);
b. selecting compounds from (a) that bind to the human RFC expressed by said cells.

27. The method of claim 26, wherein said step of contacting comprises administering said compound to said non-human animal in vivo.

28. The method of claim 27, wherein said step of selecting comprises harvesting a cell source selected from the group consisting of a cell sample, a tissue or a body fluid from said non-human animal and measuring cellular uptake of said compound by said cells.

29. The method of claim 27, wherein said step of selecting comprises harvesting a cell source selected from the group consisting of a cell sample, a tissue or a body fluid from said non-human animal and measuring binding of said compound to said human RFC expressed by said cells.

30. The method of claim 27, wherein said non-human animal has been induced to grow tumor cells, and wherein said method further comprises a step of selecting compounds from (a) that, when administered to said animal, result in a reduction in growth of said tumor cells.

31. The method of claim 26, wherein said step of contacting comprises harvesting a cell source selected from the group consisting of a cell sample, a tissue or a body fluid from said non-human animal, and contacting cells in said cell source with said compound in vitro.

32. The method of claim 26, wherein said step of selecting comprises selecting compounds from (a) that bind to the human RFC with a greater affinity than a control compound that binds to human RFC.

33. The method of claim 26, wherein the cells of said non-human animal express said human reduced folate carrier at a level that is less than the level of expression expected for expression of endogenous RFC in a wild-type animal.

34. The method of claim 26, wherein said human RFC is a mutant human RFC that is known to have reduced binding affinity for at least one known anti-folate compound.

35. A method to treat cancer, comprising administering to a patient that has cancer an anti-folate identified by the method of claim 26.

36. A method to evaluate anti-folate compounds for the treatment of cancer, comprising:
a. contacting an anti-folate compound to be evaluated with cells of a genetically modified non-human animal having human reduced folate carrier (RFC) biological activity, wherein the genome of said non-human animal comprises a first genetic modification in two alleles of its endogenous reduced folate carrier (rfc) gene, wherein said first genetic modification results in an absence of endogenous reduced folate carrier activity, and wherein the genome of said animal further comprises a second genetic modification that results in expression by said cells of said animal of a nucleic acid molecule comprising a nucleic acid sequence encoding a biologically active human reduced folate carrier (RFC);
b. selecting compounds from (a) that do not bind to the human RFC expressed by said cells, but which are internalized by said cells.

37. A method to treat cancer, comprising administering to a patient that has cancer an anti-folate identified by the method of claim 36.

38. A method for studying the molecular and biochemical events associated with folate transport, comprising:
a. harvesting cells, tissues or body fluids from a genetically modified non-human animal comprising a genetic modification in at least one allele of the endogenous reduced folate carrier (rfc) gene in said animal, wherein said genetic modification results in a reduction in endogenous reduced folate carrier (RFC) activity in said animal;
b. evaluating the cells, tissues or body fluids from the genetically modified non-human animal for molecular or biochemical activity associated with reduced folate transport in said non-human animal.

39. A method to identify compounds useful for treating a condition associated with defective folate transport in an animal, comprising:
a. contacting a compound to be evaluated with cells of a genetically modified non-human animal that has a genome comprising a genetic modification in at least one allele of the endogenous reduced folate carrier (rfc) gene in said animal, wherein said genetic modification results in a reduction in endogenous reduced folate carrier (RFC) activity in said animal;
b. selecting compounds from (a) that bind to the RFC expressed by said cells.

40. The method of claim 39, wherein said step of selecting comprises selecting compounds that bind to said RFC with a higher affinity than the affinity of folate for said RFC.

41. The method of claim 39, wherein the genome of said animal comprises a first genetic modification in two alleles of its endogenous reduced folate carrier (rfc) gene, wherein said first genetic modification results in an absence of endogenous reduced folate carrier activity, and wherein the genome of said animal further comprises a second genetic modification that results in expression by cells in said animal of a nucleic acid molecule comprising a nucleic acid sequence encoding a biologically active human reduced folate carrier (RFC).

42. The method of claim 41, wherein said step of selecting comprises selecting compounds that bind to said human RFC with a higher affinity than the affinity of folate for said human RFC.

43. The method of claim 41, wherein said human RFC is a mutant human RFC that is known to be associated with the presence of said condition.

44. The method of claim 43, wherein said compound to be evaluated is a folate analog and wherein said step of selecting comprises selecting folate analogs that bind to said mutant human RFC with an affinity that is greater than the affinity of folate for said mutant human RFC.

45. The method of claim 39, wherein said condition is a neural tube defect.

46. The method of claim 39, wherein said condition is selected from the group consisting of Parkinson's disease and Alzheimer's disease.

47. The method of claim 39, wherein said condition is a cardiovascular disease.

48. The method of claim 39, wherein said condition is stroke.

49. The method of claim 39, wherein said condition is cleft palate.

50. A method to treat a condition associated with defective folate transport, comprising administering to a patient that has said condition a compound identified by the method of claim 39.
